(19) United States
(12) Patent Application Publication
(10) Pub. No.: US 2023/0266311 A1
(43) Pub. Date: Aug. 24, 2023

(54) METHOD FOR COLLECTING OROPHARYNGEAL LAVAGE AND DETECTING ANALYTES THEREIN

(71) Applicant: ZEUS DIAGNOSTICS LLC, Severna Park, MD (US)

(72) Inventor: Raouf Giurguis, Severna Park, MD (US)

(21) Appl. No.: 18/138,563

(22) Filed: Apr. 24, 2023

Related U.S. Application Data
(63) Continuation-in-part of application No. 16/737,238, filed on Jan. 8, 2020, now Pat. No. 11,656,225.
Application No. 16/737,238 is a division of application No. 15/417,905, filed on Jan. 27, 2017, now Pat. No. 10,564,155.
Application No. 15/417,905 is a continuation-in-part of application No. 17/670,960, filed on Feb. 14, 2022.
Application No. 17/670,960 is a continuation-in-part of application No. 18/120,656, filed on Mar. 13, 2023.
(60) Provisional application No. 63/200,079, filed on Feb. 12, 2021.
(60) Provisional application No. 63/319,449, filed on Mar. 14, 2022.

(51) Int. Cl. G01N 33/543; B01L 3/00; G01N 1/18

(52) U.S. Cl. CPC G01N 33/54386; B01L 3/5023; B01L 3/5025; B01L 3/5029; G01N 1/18; G01N 2001/028

(57) ABSTRACT

A method of detecting SARS-CoV-2 virus antigen present in oropharyngeal lavage comprises: abstaining from food and drink for a first period of time before collecting the oropharyngeal lavage; pouring a non-alcoholic mouth rinse into the oral cavity, and swishing and gargling the non-alcoholic mouth rinse for a second period of time; inserting an absorbent end of a fluid collector comprising an absorbent material into the oral cavity to collect the oropharyngeal lavage from the oral cavity; removing the absorbent end of the fluid collector from the oral cavity; inserting the fluid collector into the sample receiving member of an analyte testing device for testing a fluid sample, with the absorbent end facing downward; and after a third period of time, reading a first indicator on a first test strip in the device to determine whether SARS-CoV-2 virus antigen is detected in the oropharyngeal lavage.

### CROSS-REFERENCE TO RELATED APPLICATIONS

This application is a Continuation-in-Part of U.S. Non-provisional application Ser. No. 16/737,238 filed on Jan. 8, 2020, which is a Divisional of U.S. patent application Ser. No. 15/417,905 filed on Jan. 17, 2017, now U.S. Pat. No. 10,564,155. This application is also a Continuation-in-Part of U.S. Non-provisional application Ser. No. 18/120,656 filed on Mar. 13, 2023, which claims the benefit of U.S. Provisional Patent Application No. 63/319,494 filed on Mar. 14, 2022, and a Continuation-in-Part of U.S. patent application Ser. No. 17/670,960 filed on Feb. 14, 2022, which claims the benefit of U.S. Provisional Patent Application No. 63/200,079 filed on Feb. 12, 2021, each of which are incorporated by reference herein in its entirety.

### FIELD

The present disclosure relates to liquid sample collection and testing. More particularly, the present disclosure relates to an analyte testing device that tests a fluid sample for the presence or absence of at least one analyte, secures a separate fluid sample for later confirmation, and optionally provides positive identification of an individual associated with the sample.

### BACKGROUND

Drug and other analyte testing have become ubiquitous in modern society. In homes, doctors' offices, law enforcement vehicles and offices, athletic facilities, and the workplace, effective, inexpensive and reliable testing devices have been sought. There is also a growing need for devices to test bodily fluids for substances that may assist in the diagnosis or management of diseases and other medical conditions.

The marketplace responded and is now replete with many devices directed to the testing of blood, urine or saliva. However, these devices may require a series of tests involving the shifting of the fluid sample being tested to different containers and/or the removal of the fluid sample to distant locations. These devices may also require the test administrator to handle the test subject's bodily fluids, incurring a danger of disease exposure.

Once an initial test result has been obtained, further testing of the same fluid sample to confirm or refine the initial test result is often required. For a membrane test strip device, the fluid sample may not even be retained once the initial result is obtained, necessitating retention of a split sample. The need to retain a split sample incurs the risk that a sample could be lost, mislabeled, or contaminated.

Oftentimes, the chain of custody associated with a test sample imbues the results with doubt, as the fluid sample may become contaminated, misplaced or a different fluid sample may be substituted entirely. In many instances, identification of the test subject associated with the fluid sample is critically dispositive.

Prior art testing devices include those disclosed in U.S. Pat. Nos. 7,879,623 and 8,940,527, both entitled “Integrated Device for Analyte, Testing, Confirmation, and Donor Identity Verification” and both identifying Raouf A. Guirguis as the sole inventor. U.S. Pat. Nos. 7,879,623 and 8,940,527 are both hereby incorporated by reference. The patents disclose an apparatus for fluid sample collection and analyte testing, including a single sample receiving member and at least one membrane test strip, and optionally a sample retention member, fingerprint acquisition pad, and/or fluid collector. They also provide a fluid collection apparatus having an absorbent material, compression element, and closure element, and optionally a lid that allows the apparatus to be used in conjunction with a fluid container. Also provided are methods of collecting, testing, and retaining a fluid sample and verifying the identity of one or more individuals associated with the sample, such as the test subject, test administrator, and/or witnesses. The components for collecting, testing, and retaining a fluid sample are in fluid communication with the other components of the testing device.

There is also a growing need for devices directed to testing for contaminants that may be found in food or water, such as pollutants, allergens, and harmful microbes. In some instances, it may be desirable to retain a fluid sample for confirmation testing or further analysis, retain a split fluid sample of the original sample for confirmation testing or further analysis, or to provide positive identification of the test administrator.

The Department of Transportation's (DOT) rule, 49 C.F.R. Part 40, describes required procedures for conducting workplace drug and alcohol testing for the Federally regulated transportation industry. Within this rule, definitions for split sample and split sample collection are provided. Split specimen is defined as, in drug testing, a part of the urine specimen that is sent to a first laboratory and retained unopened, and which is transported to a second laboratory in the event that the employee requests that it be tested following a verified positive test of the primary specimen or a verified adulterated or substituted test result. Split specimen collection is defined as a collection in which the urine collected is divided into two separate specimen bottles, the primary specimen (Bottle A) and the split specimen (Bottle B).

Thus, a need exists in the industry to combine the simplicity of current membrane test strip technology with the ability to positively identify the test subject and/or the test administrator, as well as the capability to secure a split portion of the fluid sample with a single device for later confirmation, within a single device.

### SUMMARY

A method of detecting SARS-CoV-2 virus antigen present in oropharyngeal lavage comprises the following steps: abstaining from placing liquid or food in an oral cavity of a user for a first period of time before collecting the oropharyngeal lavage; pouring a non-alcoholic mouth rinse into the oral cavity, and swishing and gargling the non-alcoholic mouth rinse for a second period of time to distribute the non-alcoholic mouth rinse around the inside of the oral cavity and throughout an oropharyngeal space of the user; inserting an absorbent end of a fluid collector comprising an absorbent material into the oral cavity to collect the oropharyngeal lavage from the oral cavity, the oropharyngeal lavage including the non-alcoholic mouth rinse and particles retrieved from the non-alcoholic mouth rinse; removing the absorbent end of the fluid collector from the oral cavity; inserting the fluid collector into the sample receiving member of an analyte testing device for testing a fluid sample, with the absorbent end facing downward; and after a third period of time, reading a first indicator on a first test strip in the device to determine whether SARS-CoV-2 virus antigen is detected in the oropharyngeal lavage.

The analyte testing device used in the method comprises: a sample receiving member having an opening for receiving the oropharyngeal lavage, wherein the sample receiving member includes at least first and second sample collection chambers, and a sample retention member in fluid communication with the first sample collection chamber to retain a portion of the oropharyngeal lavage; and a test cartridge member in fluid communication with only the second sample collection chamber to indicate the presence or absence of SARS-CoV-2 virus antigen in the oropharyngeal lavage; wherein the test cartridge member comprises a test cartridge having a front set of test strip slots and a back set of test strip slots, and at least one test strip to indicate the presence or absence of SARS-CoV-2 virus antigen in the oropharyngeal lavage.

A fluid collector comprises an absorbent material attached to a piston-like member, wherein the absorbent material is operatively associated with a spring such that compression of the absorbent material results in compression of the spring, and when compression is released, the spring assists return of the absorbent material to the uncompressed state.

A fluid collector comprises: an upper segment; an absorbent material to absorb an oropharyngeal lavage liquid attached to one end of the upper segment; and a storage member for holding the absorbent material and the oropharyngeal lavage liquid; wherein the oropharyngeal lavage liquid is absorbed into the absorbent material and is inserted into the storage member.

A method for collecting analyte from an oral cavity comprises: pre-soaking the absorbent material of the fluid collector of claim 28 with an oropharyngeal lavage liquid prior to insertion into the oral cavity; inserting the absorbent material of the fluid collector comprising the oropharyngeal lavage liquid into the oral cavity; ejecting the oropharyngeal lavage liquid from the absorbent material into the oral cavity; absorbing the ejected oropharyngeal lavage liquid with the absorbent material from the oral cavity; and removing the absorbent material from the oral cavity. In this method, the fluid collector can comprise an absorbent material attached to a piston-like member operatively associated with a spring such that compression of the absorbent material results in compression of the spring, and when compression is released, the spring assists return of the absorbent material to its uncompressed state; wherein the oropharyngeal lavage liquid is ejected into the oral cavity by compression of the absorbent material and spring; and wherein the oropharyngeal liquid is absorbed from the oral cavity by release of compression of the absorbent material and spring.

### BRIEF DESCRIPTION OF THE DRAWINGS

The advantages of the method disclosed herein will become more apparent by the following description and the accompanying drawings summarized below.

FIG. 1 depicts a perspective view of a fluid collection and analyte testing device in accordance with an embodiment of the present disclosure.

FIG. 2 depicts a perspective view of a fluid collection and analyte testing device in accordance with an embodiment of the present disclosure.

FIG. 3 depicts a perspective view of a fluid collection and analyte testing device in accordance with an embodiment of the present disclosure.

FIG. 4 depicts a perspective view of a housing of a fluid collection and analyte testing device in accordance with an embodiment of the present disclosure.

FIG. 5 depicts a perspective view of a test cartridge cap of a fluid collection and analyte testing device in accordance with an embodiment of the present disclosure.

FIG. 6 depicts a front view of a dual surface test cartridge with test strip holders back-to-back of a fluid collection and analyte testing device in accordance with an embodiment of the present disclosure.

FIG. 7 depicts a perspective view of a split sample (dual sample) fluid collector of a fluid collection and analyte testing device in accordance with an embodiment of the present disclosure.

FIG. 8 depicts perspective view of a fingerprint assembly of a fluid collection and analyte testing device in accordance with an embodiment of the present disclosure.

FIG. 9 depicts a front view of a dual surface test cartridge with test strips back-to-back of a fluid collection and analyte testing device in accordance with an embodiment of the present disclosure.

FIG. 10 depicts a cross-sectional end view of a housing of a fluid collection and analyte testing device in accordance with an embodiment of the present disclosure.

FIG. 11 illustrates a user swishing and gargling the non-alcoholic mouth rinse.

FIG. 12 illustrates removal of a dual-swab fluid collector from the sample receiving member of the analyte testing device.

FIG. 13 illustrates the placement of the absorbent end of a single-swab fluid collector under the user's tongue.

FIG. 14 illustrates insertion of a dual-swab fluid collector into the sample receiving member, absorbent end facing downward, until the fluid collector locks into place and the end of the fluid collector opposite the absorbent end is flush with the top of the sample receiving member.

FIG. 15 illustrates interpretation of test results from the test strips, including a positive vs. negative test result, and a valid vs. invalid test result.

FIG. 16 illustrates the function of a color indicator on a single-swab fluid collector.

FIG. 17 depicts components of a single-swab analyte testing device including a single-swab fluid collector having absorbent material, a test cartridge, and analyte testing device housing configured to receive the test cartridge with accompanying test strips and fluid collector.

FIG. 18 depicts an embodiment of a dual-swab testing device configured for testing for both SARS-CoV-2 virus antigens and SARS-CoV-2 antibodies.

FIG. 19 illustrates making a slit in the test cartridge cap.

FIG. 20 depicts an embodiment of the analyte testing device in which the sample receiving member is integrated into the test cartridge.

FIG. 21 depicts an embodiment of a dual-swab fluid collector in which the absorbent material is pre-soaked and stored with an oropharyngeal lavage liquid in a collection housing member.

FIG. 22 depicts components of an embodiment of a fluid collector in which the absorbent material is operatively associated with a spring such that compression of the absorbent material results in compression of the spring, and when compression is released, the spring assists return of the absorbent material to the uncompressed state.

### DETAILED DESCRIPTION

The present method provides a rapid and effective screening test for a variety of analytes, such as SARS-CoV-2 virus, Herpes simplex virus, Epstein-Barr virus, cytomegalovirus, Moraxella catarrhalis, Norocardia sp., Streptococcus mutans, Streptococcus oralis, Eikenella sp., Neisseria sp., and Streptococcus salivarius. The method utilizes the collection of oropharyngeal lavage, vertical flow test strips for chromatographic immunoassays, for the simultaneous, qualitative or quantitative detection of analytes in oropharyngeal lavage. Advantageously, compared to nasopharyngeal swabs, collection of oropharyngeal lavage is simple, easy, and comfortable, and does not require trained medical personnel to take the sample. Therefore it can be performed in the home by the test subject or family member. A further advantage of the method is that the virus may be detected earlier than with a nasopharyngeal swab, because the amount of virus can peak in the oral mucosa and saliva before peaking in the nasopharynx. Moreover, the non-alcoholic mouth rinse breaks up the bacterial and viral contents of the user's mouth, thereby releasing biological analytes that can be detected by the test, for example bacterial and viral antigens. In addition, the non-alcoholic mouth wash disinfects the sample. For example, test results have shown that 99.99% of virus samples are disinfected by OPL™ non-alcoholic mouth rinse. Advantageously, this reduces the spread of the SARS-CoV-2 infection between individuals during testing procedures.

As used herein, “oral cavity” includes the lips, the inside lining of the lips and cheeks (buccal mucosa), the teeth, the gums, the front two-thirds of the tongue, the floor of the mouth below the tongue, the bony roof of the mouth (hard palate) and the area behind the wisdom teeth (called the retromolar trigone). As used herein, the term “mouth” is synonymous with “oral cavity”. The oropharynx is the middle part of the throat just behind the oral cavity, and defines the oropharyngeal area. It can be seen when your mouth is wide open. It includes the base of the tongue (the back third of the tongue), the soft palate (the back part of the roof of the mouth), the tonsils, and the side and back walls of the throat. The oral cavity and oropharynx help you breathe, talk, eat, chew, and swallow. Minor salivary glands all over the oral cavity and oropharynx make saliva (spit) that keeps your mouth and throat moist, and helps you digest food. The oral mucosa is the mucous membrane lining the inside of the oral cavity and the oropharynx. The oral mucosa and secreted saliva can harbor viruses such as the SARS CoV-2 virus.

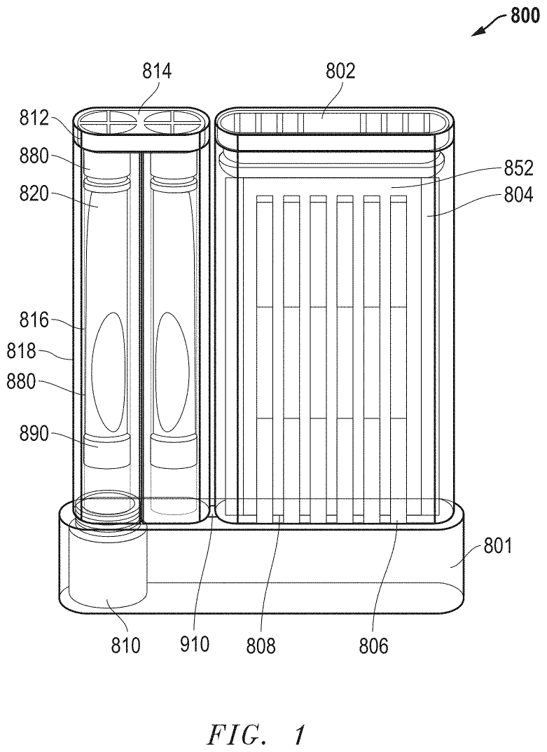

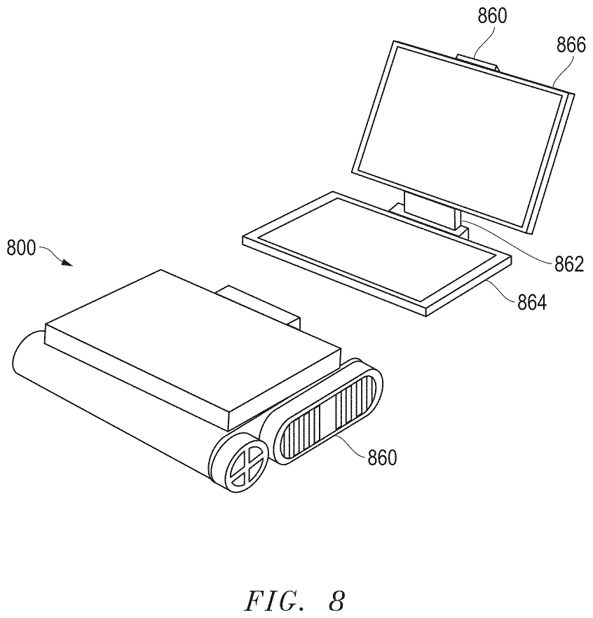

With reference to FIG. 1, the analyte testing device 800 used in the method includes at least one membrane test strip 806 in fluid communication with a sample receiving member 818, where the test strip is configured to indicate the presence or absence of at least one analyte above or below a threshold concentration in oropharyngeal lavage using a vertical flow chromatographic assay. The device 800 includes a receiving member having an opening to receive a fluid sample. The sample receiving member 818 is in fluid communication with other components of the device, a sample retention member, such as confirmation collection chamber 810, a test cartridge member 804, including at least one membrane test strip 806, and optionally an immunoassay-based fingerprint acquisition pad 860. (See FIG. 8.) Fluid communication within the device can be via channels, e.g. tubes, piping, channels molded or carved into the device, or any other suitable structure, made of any suitable material, for example plastic, ceramic, metal, glass, wood, rubber, polymers, fiber-reinforced polymer, or any combination thereof. The analyte test device 800 is configured to include a sample retention member having a confirmation collection chamber 810 in order to retain at least a volume of oropharyngeal lavage obtained from the user. The sample retention member can comprise an absorbent material to soak up the oropharyngeal lavage. The retained portion of the oropharyngeal lavage can be used for further testing, for example for confirmation of a test result obtained using a membrane test strip, or to test for the presence or absence of other analytes in the fluid sample. The retained portion of the fluid sample can also be used for confirmation of the test subject's identity through analysis of a distinguishing feature of the subject, including without limitation DNA, cells, proteomics, metals, and liquid biopsies.

### Analyte Screening

An embodiment of the present method provides an analyte screening device which includes a rapid screening, lateral flow or vertical flow chromatographic immunoassay for the simultaneous, qualitative or quantitative detection of analytes in a fluid sample. For example, without limitation, the fluid sample may be saliva, urine, blood, mucus, water, or fluid extract of a solid or a semi-solid, for example stool, mucus, or liquid biopsy. The fluid sample may also be an environmental sample, for example, without limitation, soil, dust, water, plant matter, insect matter, animal matter, or a fluid extract of any of the foregoing. The fluid sample may also be a food or beverage, for example, without limitation, a liquid beverage, a liquid-containing food, or a fluid extract of a solid, semi-solid or powdered food or beverage. The fluid sample may also contain genomic or proteomic material for testing and analysis.

Some embodiments include at least one membrane test strip, in fluid communication with a sample receiving member, able to indicate the presence or absence of at least one analyte above or below a threshold concentration in the fluid sample using a lateral flow or vertical flow chromatographic assay. “Membrane test strip” and “test strip” are used interchangeably herein.

In some embodiments, the lateral flow or vertical flow chromatographic assay is a competitive assay, in which an analyte in the fluid sample competes with a competitor for binding with an anti-analyte antibody. For example, the anti-analyte antibody may be labeled, and the competitor may be immobilized in the test region of the membrane test strip. After the fluid sample reaches the dye region, it encounters the labeled anti-analyte antibody. If the analyte is present in the fluid sample above a predetermined threshold concentration, the analyte will saturate the binding sites of the labeled anti-analyte antibody; otherwise, some or the entire labeled anti-analyte antibody remains free to bind the competitor. As the fluid sample migrates along the membrane test strip by capillary action, it carries the labeled anti-analyte antibody along until it reaches the test region. The test region contains the immobilized competitor, which may be the analyte, fragments of the analyte, epitopes of the analyte, molecular mimics of the analyte, anti-idiotypic antibodies, or any other molecule able to compete with the analyte for binding to the anti-analyte antibody. If the analyte is present above the predetermined threshold concentration, the labeled anti-analyte antibody is saturated and does not bind the immobilized competitor, resulting in no signal in the test region; otherwise, the anti-analyte antibody is unsaturated and can bind to the competitor, resulting in a signal in the test region.

Thus, according to an embodiment of the disclosure, employing a competitive assay, an analyte-negative fluid sample (containing lower than the predetermined concentration of the analyte) will generate a line in the test region due to capture of the labeled anti-analyte antibody, whereas an analyte-positive fluid specimen will not generate a colored line in the test region because the analyte in the fluid sample will saturate the labeled antibody and thus prevent its capture in the test region.

In an embodiment of the disclosure, the lateral flow or vertical flow chromatographic assay is a sandwich assay, in which the analyte must be present for the labeled anti-analyte antibodies to be captured in the test region. For example, the analyte antibody may be a labeled antibody, and a second anti-analyte antibody may be immobilized in the test region. For example, after the fluid sample reaches the dye region, it encounters the labeled anti-analyte antibody. If the analyte is present in the fluid sample, it will bind at least a fraction of the labeled anti-analyte antibody. As the fluid sample migrates along the membrane test strip by capillary action, it carries the labeled anti-analyte antibody along until it reaches the test region. The test region contains an immobilized anti-analyte antibody, which may be reactive against a different epitope of the analyte than the labeled anti-analyte antibody. If the analyte is present in the fluid sample, it forms a scaffold through which the labeled antibodies are immobilized in the test region. The fraction of the labeled antibodies captured in the test region is thus determined by the concentration of analyte in the fluid sample. If the analyte of interest is present above a predetermined threshold concentration, a sufficient fraction of the labeled antibodies are captured, resulting in a visible signal in the test region; otherwise, an insufficient fraction of the antibodies are captured and no signal is visible in the test region.

Thus, according to an embodiment of the disclosure employing a sandwich assay, an analyte-positive fluid specimen will generate a colored line in the test region of the membrane test strip due to the capture of the labeled antibody in the test region, whereas an analyte-negative fluid sample will not generate a fine in the test region due to failure to capture the labeled antibody.

Embodiments of the disclosure include a positive control to indicate that the assay has functioned properly and is complete. For example, the dye region may include a labeled control protein, including without limitation a labeled control antibody, and the control region of the membrane test strip may contain an immobilized control agent able to capture the labeled control protein, such as an antibody or a control analyte. The control region may be located distal to each test region on the membrane test strip, such that the fluid sample will encounter each test region before encountering the control region. The reaction of the labeled control protein with the immobilized control agent produces a colored line in the control region, indicating that a proper volume of the fluid sample has been added and membrane wicking has occurred, and the assay has worked properly.

An embodiment of the disclosure concurrently tests for multiple analytes, for example by employing membrane test strips capable of testing multiple analytes concurrently (for example, by containing multiple anti-analyte antibodies in the dye region and having multiple compatible test region), and/or by employing multiple membrane test strips within the same device. An embodiment of the disclosure includes both membrane test strips that employ a competitive assay and a sandwich assay, for example on different membrane test strips within the device and/or on the same membrane test strip within the device.

Embodiments of the disclosure may provide quantitative determination of the concentration of an analyte that is present in the fluid sample. For example, the apparatus may include multiple membrane test strips having varying amounts of an anti-analyte antibody, resulting in varying analyte sensitivity, such that the concentration of the analyte is indicated by which of the membrane test strips show or fail to show a colored line in the test region.

### Antibodies

An embodiment of the disclosure employs antibodies for the detection of analytes. The term “antibody” (Ab) as used herein includes monoclonal antibodies, polyclonal antibodies, multispecific antibodies (for example bispecific antibodies), and antibody fragments, so long as they exhibit the desired activity. The term “monoclonal antibody” as used herein refers to an antibody obtained from a population of substantially homogeneous antibodies, that is, the individual antibodies comprising the population are identical except for possible naturally occurring mutations that may be present in minor amounts.

The terms “labeled antibody” and “labeled control protein” refer to an antibody or protein that is conjugated directly or indirectly to a label. The label is a detectable compound or composition that may be detectable by itself, including without limitation a dye, colloidal metal (including without limitation colloidal gold), a radioisotope, or a fluorescent compound, or, in the case of an enzymatic label, may catalyze chemical alteration of a substrate compound or composition which is detectable, or any combination of the foregoing.

### Analytes

According to an embodiment of the disclosure, an apparatus includes an analyte testing device, which is a device for testing a fluid sample for the presence of analytes. The present disclosure contemplates testing for any analyte. Without limitation, analytes that may be tested for include drugs of abuse or their metabolites, analytes indicating the presence of an infectious agent or product of an infectious agent, allergens, pollutants, toxins, contaminants, analytes with diagnostic or medical value, antibodies against any of the foregoing, and any combination thereof.

According to an embodiment of the disclosure, analytes that may be tested for include drugs of abuse and their metabolites, including without limitation 7-acetaminoclonazepam, alkyl nitrites, alpha-hydroxyalprazolam, alprazolam, 2-amino-2′-chloro-5-nitrobenzophenone, 7-aminoclonazepam, 7-aminonitrazepam, amitriptyline, amobarbital, amoxapine, amphetamine, anabolid steroids, androgen, androstadienone, aprobarbital, atropine, barbiturates, benzodiazepines, benzoylecgonine, benzylpiperazine, boldenone undecylenate, 4-bromo-2,5-dimethoxyphenethylamine, bovine growth hormone, butabarbital, butalbital, butripryline, 4-chlordehydromethyltestosterone, chloroform, clomipramine, clonazepam, clostebol, cocaethylene, cocaine, codeine, codeine-6-glucuronide, cotinine, dehydroepiandrosterone, desipramine, desmethyldiazepam, desoxymethyltestosterone, dexmethylphenidate, dextroamphetamine, dextromethorphan, dextropropoxyphene, dextrorphan, 2,5-diamino-T-chlorobenzophenone, diamorphine, diazepam, dibenzepin, dihydrotestosterone, dimenhydrinate, 2,5-dimethoxy-4-(n)-propylthiophenethylamine, 2,5-dimethoxy-4-ethylphenethylamine, 2,5-dimethoxy-4-iodophenethylamine, dimethyl ether, dimethyltryptamine, dimethyltryptamine, diphenhydramine hydrochloride, dosulepin hydrochloride, dothiepin hydrochloride, doxepin, drostanolone, ecgonine, ecgonine methyl ester, ephedrine, ergine, estren, 5-estrogen, ethyl-5-(1′-methyl-3′-carboxypropyl)-2-thiobarbituric acid, 5-ethyl-5-(1′-methyl-3′-hydroxybutyl)-2-thiobarbituric acid, ethylestrenol, ethylphenidate, fentanyl, flunitrazepam, fluoxymesterone, furazabol, gamma-hydroxybutyrate, 1-(beta-D-glucopyranosyl) amobarbital, growth hormone, heroine, hexabarbital, human chorionic gonadotropin, human growth hormone, hydrocodone, hydromorphone, (+)-3-hydroxy-N-methylmorphinan, 3-hydroxy clonazepam, 11-hydroxy-tetrahydrocannabinol (11-hydroxy-THC), 3′-hydroxyamobarbital, p-hydroxyamphetamine, p-hydroxynorophedrine, imipramine, iprindole, kava, ketamine, levomethylphenidate, iofepramine, lorazepam, lorazepam-glucuronide, lysergic acid diethylamide, meperidine, mescaline, mestanolone, mesterolone, meta-chlorophenylpiperazine, methadone, methamphetamine, methandrostenolone, methcathinone, 3,4-methylenedioxyamphetamine, methanolone, methanolone enanthate, methylenedioxymethamphelamine (ecstacy), methylphenidate, methylphenobarbital, methyl testosterone, mibolerone, (+)-3-morphinan, morphine, nandrolone, nicotine, nitrazepam, N-methyl-diethanciamine, norbolethone, norcodeine, norethandrolone, norketamine, nortriptyline, opiates, opipramol, opium, oxabolone opionate, oxandrolone, oxazepam, oxycodone, oxymetholone, oxymorphone, pentobarbital, phencyclidine, phenethylamines, phenobarbital, 4-phenyl-4-(1-piperidinyl)-cyclohexanol, 1-phenyl-1-cyclohexene, phenylacetone, 5-[N-(1-phenylcyclohexyl)]-aminopentanoic acid, 1-(1-phenylcyclohexyl)-4-hydroxypiperidine, piperidine, protriptyline, psilocin, psilocybin, quinbolone, salvinorin A, scopolamine, secobarbital, sodium thiopental, stanozolol, telbutal, temazepam, testosterone, testosterone propionate, tetrahydrocannabinol (THC), THC-COOH, tetrahydrogestrinone, toluene, trenbolone, tricyclic antidepressants, 3-trifluoromethylphenylpiperazine, trimipramine, tryptamines, or any combination thereof. The minimum concentration level at which the presence of any particular drug or metabolite is detected may be determined by various industry minimum standards, such as, for example, the National Institute on Drug Abuse (NIDA), the Substance Abuse & Mental Health Services Administration (SAMHSA), and the World Health Organization (WHO).

According to an embodiment of the disclosure, analytes that may be tested for include infectious agents or the products of an infectious agent, including without limitation acanthamoeba, aflatoxin, alimentary mycotoxlcoses, altertoxin, amoeba, anisakis, Ascaris lumbricoides, Bacillus arthracis, Bacillus cereus or its toxin, bacteria, bovine spongiform encephalopathy prioris, Brucella, Caliciviridae, Calymmatobacterium granulomatis, Campylobacter, Campylobacter jejuni, Candida, Candida albicans, Cephalosporium, Chlamydia trachomatis, chronic wasting disease prions, Citrinin, Clostridium botulinum or its toxin, Clostridium perfringens, Corynebacterium ulcerans, Coxielia burnetil, Creutzieldt-Jakob disease prions, Cryptococcus neoformans, Cryptosporidium, Cryptosporidium parvum, cyclopiazonic acid, Cyclospora cayetanensis, cytochalisins, cytomegalovirus, Diphyilobothrium, Escherichia coli, Ebola, endotoxins, Entamceba histolytica, enteroviruses, ergopeptine alkaloid, Ergot alkaloid, ergotamine, Escherichia coli O157, Eustrongylides, Fasciola hepatica, fatal familial insomnia prions, flatworm, Francisella tularensis, fumitremorgen B.sub.1, fumonisin, Fusarium, fusarochromanone, genital warts, Gerstmann-Straussler-Scheinker syndrome prions, Giardia, Giardia lamblia, Granuloma inguinale, H7 enterohemorrhagic E. coli, Haemophilus ducreyl, Helicobacter pylori, Hepatitis, Hepatitis A, Hepatitis B, Hepatitis C, Hepatitis D, Hepatitis E, Hepatitis E, herpes simplex virus, Histoplasma capsulatum, HIV, HIV-1, HIV-2, human papillomavirus, influenza, Kaposi's sarcoma-associated herpesvirus, kojic acid, kuru prions, Listeria monocytogenes, lolitrem alkaloids, marburg virus, methicillin-resistant Staphylococcus aureus or its toxin, molluscum, moniliformin, mononucleosis, mycobacteria, Mycobacterium tuberculosis, Mycoplasma, Mycoplasma hominis, mycotoxins, Myrothecium, Nanophyetus, Neisseria gonorrhoeae, nematodes, nivalenol, norovirus, ochratoxins, oosporeine, parasites, patulin, paxilline, penitrem A, phomopsins, Plasmodium, Platyhelminthes, Plesiomonas shigelloides, pneumococcus, Pneumocystis jirovecii, prions, protozoa, rhinovirus, rotavirus, Salmonella, Sarcocystis hominis, Sarcocystis sulhominis, scrapie prions, sexually transmitted diseases, Shigella, sporidesmin A, Stachybotrys, Staphylococcus aureus or its toxin, sterigmatocystin, Streptococcus, Streptococcus pneumoniae, Streptococcus pyogenes, Taenia saginata, Taenia solium, tapeworms, Tenia solium, tinea, Toxoplasma gondii, tremorgenic mycotoxins, Treponema palidum, Trichinella spiralis, Trichoderma, Trichomonas vaginalis, trichothecenes, Trichuris trichlura, Typanosoma cruzi, Ureaplasma urealyticum, verrucosidin, verruculogen, Vibrio cholerae non-O1, Vibrio cholerae O1, Vibrio parahaemolyticus, Vibrio vulnificus, viruses, yeast infections, Yersinia enterocolitica, Yersinia pseudotuberculosis, Zearalenois, zearalenone, antibodies against any of the foregoing, or any combination thereof.

According to an embodiment of the disclosure, the analytes to be tested for include allergens, including without limitation Aesculus, aider, almonds, animal products, Arternisia vulgaris, beans, bet sting venom, birch, calyx, cat dander, celeriac, celery, Chenopodium album, cockroach, corn, dander, dong dander, drugs, dust mite excretions, egg albumen, eggs, Fel d 1 protein, fruit, fur, grass, hazel, hornbeam, insect stings, latex, legumes, local anaesthetics, maize, metal, milk, mold spores, mosquito saliva, mouse dander, nettle, Olea, peanuts, peas, pecans, penicillin, plant pollens, Plantago, Platanus, poplar, pumpkin, ragweed, rat dander, ryegrass, salicylates, seafood, sesame, sorrel, soy, soybeans, sulfonamides, Tilia, timothy-grass, tree nuts, trees, wasp sting venom, weeds, wheat, willow, antibodies against any of the foregoing, or any combination thereof.

According to an embodiment of the disclosure, the analytes to be tested for include pollutants, toxins, and contaminants, including without limitation 1,2-dibromoethane, acrylamide, aldehydes, arsenic, artificial growth hormone, asbestos, benzene, benzopyrene, carcinogens, dichloro-diphenyl-trichloroethane, formaldehyde, KEPONE™, lead, mercury, methylmercury, nitrosamines, N-nitroso-N-methylurea, organochlorine insecticides, pesticides, polychlorinated biphenyls, polychlorinated dibenzofurans, polychlorinated dibenzo-p-dioxins, recombinant bovine growth hormone, recombinant bovine somatotropin, toluene, vinyl chloride, antibodies against any of the foregoing, or any combination thereof.

According to an embodiment of the disclosure, the analytes to be tested for include analytes with diagnostic or medical value, including without limitation acid phosphatase, active-B12, AFP, alanine aminotransferase, alanine aminotransferase, albumin, albumin BCG, albumin BCP, alkaline phosphatase, alpha-1 antitrypsin, alpha-1 glycoprotein, amikacin, ammonia, amylase, anti-CCP, anti-Tg, anti-TPO, apolipoprotein A1, apolipoprotein B, ASO, aspartate aminotransferase, B12, beta 2 microglobulin, beta 2 microglobulin, BNP, CA 125, CA 125 II, CA 15-3, CA 19-9 XR, calcium, carbamazepine, carbon dioxide, CEA, ceruloplasmin, cholesterol, CK-MB, complement C3, complement C4, cortisol, C-peptide, C-reactive protein, creatine kinase, creatinine, cyclosporine, cyclosporine and metabolite-whole blood, cyclosporine monoclonal-whole blood, D-dimer, DHEA-S, digitoxin, digoxin, digoxin II, digoxin III, direct bilirubin, direct LDL, estradiol, ferritin, FLM II, folate, free carbamazepine, free phenytoin, free PSA, free T3, free T4, free valproic acid, FSH, gamma-glutamyl transferase, gentamicin, glucose, glycated hemoglobin, haptoglobin, hCG, hemoglobin, homocysteine, ICT CI-, IGFBP-1, immunoglobulin, immunoglobulin A, immunoglobulin E, immunoglobulin G, immunoglobulin M, insulin, intact PTH, Iron, K⁺, kappa light chain, lactate dehydrogenase, lactic acid, lambda light chain, LH, lidocaine, lipase, lithium, Lp, magnesium, metabolites, methotrexate II, microalbumin, MPO, myoglobin, Na⁺, N-acetyl-procainamide, neonatal bilirubin, NGAL, P-amylase, pepsinogen I, pepsinogen II, phenobarbital, phenytoin, phosphorus, prealbumin procainamide, progesterone, prolactin, quinidine, rheumatoid factor, SHBG, sirolimus, STAT CK-MB, T4, tacrolimus, tacrolimus II, testosterone, Tg, theophylline, theophylline II, TIBC, TIMP-1, tobramycin, total bilirubin, total estriol, total protein, total PSA, total T3, total T4, transferrin, triglycerides, troponin-I, troponin-I ADV, TSH, T-uptake, UIBC, ultra HDL, urea nitrogen, uric acid, urine/CSF protein, valproic acid, vancomycin, vancomycin II, vitamin D, antibodies against any of the foregoing, or any combination thereof.

### Receiving Member

According to an embodiment or the disclosure, the apparatus includes a receiving member, having an opening to receive a fluid sample. For example, the receiving member may be dimensioned to receive a fluid collector. In an embodiment of the disclosure, the receiving member may be in fluid communication with other components of the apparatus, for example at least one membrane test strip, sample retention member, and/or an immunoassay-based fingerprint acquisition pad, through channels, for example tubes, piping, channels molded or carved into the apparatus, or any other suitable structure, made of any suitable material, for example plastic, ceramic, metal, glass, wood, rubber, polymer, fiber-reinforced polymer, or any combination thereof.

According to an embodiment of the disclosure, the channel or channels providing fluid communication between the components may have differing flow resistance, for example having channels, channel segments, or openings, that are narrower, wider, longer, or shorter than others, and/or having fluid paths with varying amounts of vertical rise or drop, such that the fluid channels within the device have varying degrees of flow resistance. For example, the channel that provides the fluid communication of the sample receiving member with the at least one membrane test strip may have greater flow resistance than the at least one channel that provides the fluid communication of the sample receiving member with the sample retention member, to ensure that a portion of the fluid sample is collected in the sample retention member.

In an embodiment of the disclosure, a single channel having multiple openings may connect the receiving member to each of the components of the apparatus with which it is in fluid communication, for example the at least one membrane test strip, sample retention member, and/or immunoassay-based fingerprint acquisition pad.

In an embodiment of the disclosure, the receiving member may include two or more chambers for receipt of a multi-pronged fluid collector, including but not limited to a dual-swab fluid collector. Components of the analyte testing device may be solely connected to one of the multiple chambers. For example, in a two-chamber embodiment, one chamber may be solely connected to a sample retention member to ensure that a portion of the fluid sample is collected and stored without interaction of the other components of the device.

An embodiment of the disclosure may accommodate fluids of varying viscosity, for example water, saliva, urine, blood, oropharyngeal lavage, and liquids associated with genomics and proteomics. Generally, this is accomplished by varying the diameter of the channel or channels that provide the fluid communication of the sample receiving member with the other components of the device, for example providing a wider channel diameter to accommodate a more viscous fluid.

In an embodiment of the disclosure, a channel dimensioned to be compatible with a fluid having the viscosity of water provides the fluid communication of the sample receiving member with the at least one membrane test strip; and at least one channel dimensioned to be compatible with a fluid having the viscosity of water provides the fluid communication of the sample receiving member with the sample retention member.

In an embodiment of the disclosure, a channel dimensioned to be compatible with a fluid having the viscosity of urine provides the fluid communication of the sample receiving member with the at least one membrane test strip; and at least one channel dimensioned to be compatible with a fluid having the viscosity of urine provides the fluid communication of the sample receiving member with the sample retention member.

In an embodiment of the disclosure, a channel dimensioned to be compatible with a fluid having the viscosity of saliva provides the fluid communication of the sample receiving member with the at least one membrane test strip; and at least one channel dimensioned to be compatible with a fluid having the viscosity of saliva provides the fluid communication of the sample receiving member with the sample retention member.

In an embodiment of the disclosure, a channel dimensioned to be compatible with a fluid having the viscosity of blood provides the fluid communication of the sample receiving member with the at least one membrane test strip; and at least one channel dimensioned to be compatible with a fluid having the viscosity of blood provides the fluid communication of the sample receiving member with the sample retention member. In an embodiment of the disclosure, a channel dimensioned to be compatible with a fluid having the viscosity of mucus provides the fluid communication of the sample receiving member with the at least one membrane test strip; and at least one channel dimensioned to be compatible with a fluid having the viscosity of mucus provides the fluid communication of the sample receiving member with the sample retention member.

In an embodiment of the disclosure, a channel dimensioned to be compatible with a fluid having the viscosity of liquid associated with cell separation provides the fluid communication of the sample receiving member with the at least one membrane test strip; and at least one channel dimensioned to be compatible with a fluid having the viscosity of liquid associated with cell separation provides the fluid communication of the sample receiving member with the sample retention member.

In an embodiment of the disclosure, a channel dimensioned to be compatible with a fluid having the viscosity of liquid biopsy, such as proteomics or genomics, provides the fluid communication of the sample receiving member with the at least one membrane test strip; and at least one channel dimensioned to be compatible with a fluid having the viscosity of liquid biopsy provides the fluid communication of the sample receiving member with the sample retention member. Proteomics is the study of proteins. Genomics is a branch of molecular biology concerned with the structure, function, evolution, and mapping of genomes.

In an embodiment of the disclosure, a channel dimensioned to be compatible with a fluid having the viscosity of oropharyngeal lavage provides the fluid communication of the sample receiving member with the at least one membrane test strip; and at least one channel dimensioned to be compatible with a fluid having the viscosity of oropharyngeal lavage provides the fluid communication of the sample receiving member with the sample retention member.

In an embodiment of the disclosure, the receiving member may have an inner surface, for example a lower surface, that an absorbent material, such as an absorbent material present in a fluid collector, may be compressed against, thereby expelling the fluid sample from the absorbent material. For example, the absorbent material may be compressed directly between a compression member present on the fluid collector and the lower surface of the receiving member or the receiving member may provide structural support to facilitate compression of the absorbent material between a compression member and the housing that at least partially surrounds the absorbent material.

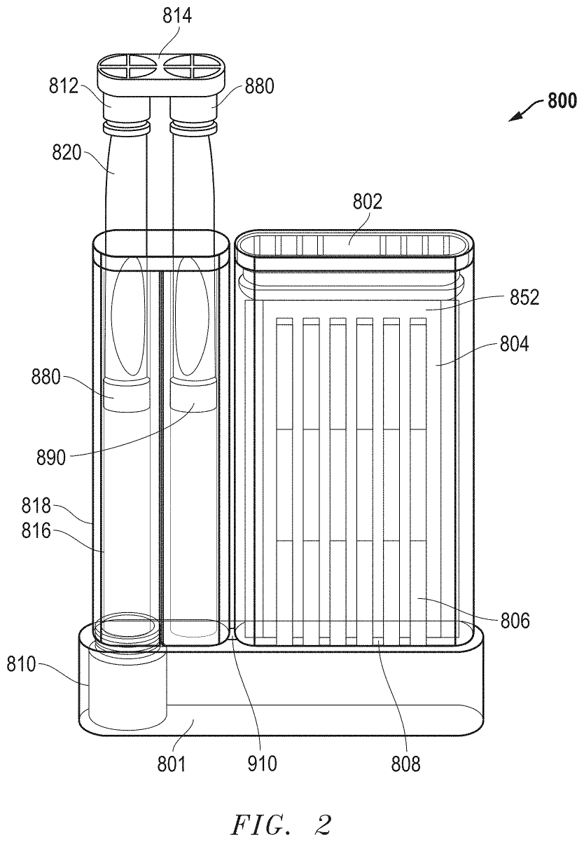

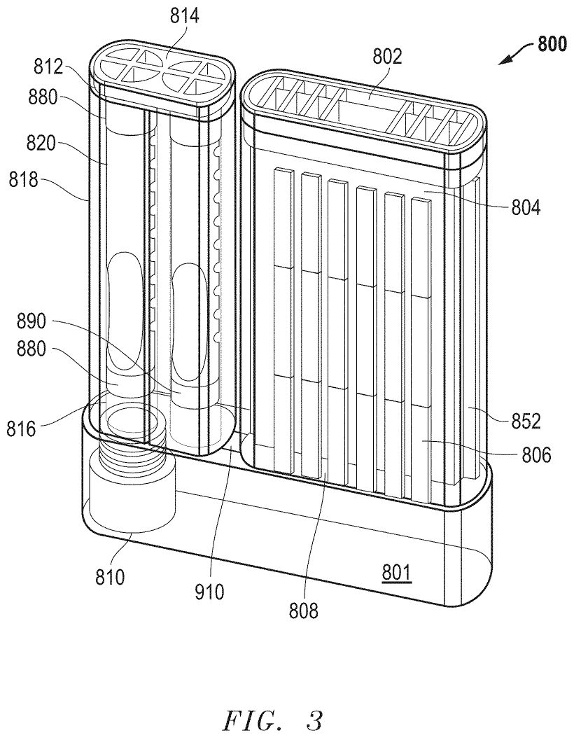

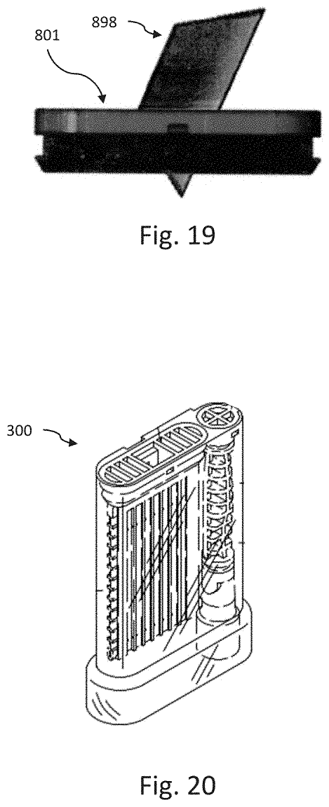

In the embodiments depicted in FIG. 1-4, the sample receiving member 818 is separated from the test cartridge 804. In these embodiments, a means for release or air pressure when the test cartridge cap 802 is inserted into the test cartridge member 804 may be needed. In these embodiments, a slit is made in the test cartridge cap 802 for release of any pressure build-up. The slit can be made, for example by using a box cutting blade 898 as depicted in FIG. 19. In some embodiments, the sample receiving member is integrated into the test cartridge, for example as disclosed in U.S. Patent Application Publication No. US 2019/0049442 A1, which is incorporated herein by reference in its entirety. In these embodiments, a means for release of air pressure may not be necessary. An example of an analyte testing device design 300 in which the sample receiving member is integrated into the test cartridge is depicted in FIG. 20.

### Sample Retention Member

According to an embodiment of the disclosure, the analyte testing device includes a sample retention member. The sample retention member may be used to securely contain a portion of the fluid sample, such as a split sample. The retained portion of the fluid sample may be used for further testing, for example for confirmation of a test result obtained using a membrane test strip, or to test for the presence or absence of other analytes in the fluid sample. The retained portion of the fluid sample may also be used for confirmation of the test subject's identity through analysis of a distinguishing feature thereof, including without limitation DNA, cells, proteomics, metals, and liquid biopsy.

According to one embodiment of the disclosure, the sample retention member includes an absorbent material, for example a pad or sponge, or made of woven or non-woven fibrous or fabric-like material, for example cellulose or a cellulose derivative, cotton, hydrophilic foam, wood pulp, polyvinyl alcohol fibers, or any combination thereof. The sample retention member may include an absorbent material that is part of the sample collection apparatus. The absorbent material may be surrounded by a barrier, such as a liquid-impermeable material, including without limitation plastic, ceramic, metal, glass, wood, rubber, polymer, fiber-reinforced polymer, or any combination thereof, to prevent the retained sample from leaking or evaporating. In an embodiment of the disclosure, the absorbent material may be removably attached to the apparatus to facilitate retrieval of the retained fluid sample. In an embodiment of the disclosure, the absorbent material may be accessed using a needle, for example by piercing a barrier surrounding the absorbent material. The retained sample may then be removed, for example, into a syringe attached to a needle, by means of withdrawal of the syringe to create suction.

According to an embodiment of the disclosure, the sample retention member includes a storage container defining a volume for storage of the fluid sample. In one embodiment of the disclosure, the sample retention member may be a vial made from a breakable or nearly unbreakable material, including without limitation glass, plastic, ceramic, metal, metal foil, wood, rubber, polymer, fiber-reinforced polymer, or any combination thereof. In an embodiment of the disclosure, the storage container may be accessed using a needle to pierce the wall of the storage container. For example, the storage container may include a pierceable member, such as a region of decreased wall thickness, and/or made of a soft, pierceable, or breakable material, including without limitation plastic, ceramic, metal, glass, metal foil, wood, rubber, polymer, fiber-reinforced polymer, or any combination thereof, that may be pierced. The retained sample may then be removed, for example, into a syringe attached to a needle, by means of withdrawal of the syringe to create suction. In an embodiment of the disclosure, the storage container may be removably attached to the apparatus, including without limitation, through a line of weakness that may allow the storage container to be broken free form the apparatus, through a threaded connection mechanism between the sample retention member and the fluid sample testing device, or through a twisting lock connection mechanism between the sample retention member and the fluid sample testing device.

According to an embodiment of the disclosure, the removable sample retention member may be linked to or coded consistently with the analyte testing device by, for example, identical or related identification or serial numbers on both the sample retention member and the fluid sample testing device, by identical or related bar code information on both the sample retention member and the fluid sample testing device, and/or by the inclusion of radio frequency identification (RFID) devices on the sample retention member or the sample retention member and the fluid sample testing device. RFID incorporates the use of electromagnetic or electrostatic coupling in the radio frequency (RF) portion of the electromagnetic spectrum uniquely identify an object; such unique identification information may be information specific to the sample provider or information unique to the fluid sample testing device.

According to an embodiment of the disclosure, the sample retention member contains substances that facilitate a further use of the sample, including without limitation preservatives or stabilizers able to preserve sample integrity, for example substances able to inhibit microbial growth, kill microbes, prevent sample leakage, prevent sample evaporation, inhibit chemical or enzymatic degradation of substances in the sample, support survival of cells or other microbes in the sample, or any combination thereof.

According to an embodiment of the disclosure, the sample retention member may be bonded to a fingerprint acquisition pad. For example, such a bond may provide a safeguard against dissociation of the retained sample from the fingerprint.

According to an embodiment of the disclosure, the sample retention member may be in fluid contact solely with the sample receiving member and may not have any fluid contact with any other component of the device.

The retained fluid sample may be used for further confirmation testing, including without limitation gas chromatography, liquid chromatography, mass spectrometry, liquid or gas chromatography with tandem mass spectrometry, polymerase chain reaction, DNA sequencing, enzyme-linked immunosorbent assay (ELISA), western blotting, culturing for growth, or any combination thereof, using the retained fluid sample.

### Fluid Collector

An embodiment of the analyte testing device comprises a fluid collector for collecting a fluid sample. The present disclosure contemplates collecting a sample from a specific subject, such as a human subject, environmental samples, such as air, water, or soil, some other substance, such as a food or beverage, or a liquid extract of any of the foregoing. The fluid collector is operatively associated with the device. The fluid collector may be removably associated with the device, affixed to the device, or comprise multiple units of which one or more is affixed or removably associated with the device.

In an embodiment of the disclosure, the fluid collector includes an absorbent material or swab capable of absorbing a desired quantity of a fluid sample. The absorbent material may be made of any suitable material known to a person in the art, for example, without limitation, a pad, sponge, or woven or non-woven fibrous or fabric-like material, including without limitation cellulose or cellulose derivative, cotton, hydrophilic foam, wood pulp, polyvinyl alcohol fibers, or any combination thereof. In an embodiment of the disclosure, the fluid collector includes a compression member, able to compress the absorbent material, that may be used to expel air from the absorbent material prior to collection of the fluid sample and/or encourage the fluid sample to flow into the absorbent material by creating suction as the compressed absorbent material returns to the uncompressed state. A compression member may also be used, for example, to compress the absorbent material and expel a fluid sample contained therein.

In some embodiments of the disclosure, the fluid collector includes multiple collection swabs. For example, a two-prong fluid collector with dual swabs may be implemented to collect the sample. In some embodiments, each swab of a multi-swab fluid collector is selected based upon the specific swab collection characteristics. For example, in a dual-swab fluid collector, each swab may contain a material to assist in the collection of different samples such as the collection of different cell material.

A sufficiency indicator on the collector is contemplated. For example, a color indicator may either appear or disappear when a sufficient sample has been collected, for example when a sufficient volume has been absorbed to reach the location in the absorbent material where the sufficiency indicator is disposed. According to an embodiment of the disclosure, the sufficiency indicator may be operatively associated with the absorbent material and may be protected from direct contact with the source of the fluid sample by a barrier, such as a transparent barrier, for example plastic or glass, such that the fluid sample will only reach the sufficiency indicator by passing into the absorbent material.

The sufficiency indicator color may be in the shape of a word or symbol that appears or disappears when a sufficient sample has been collected. For example, the sufficiency indicator may be a diffusible dye, wherein dilution of the diffusible dye by the fluid sample causes a color to disappear, indicating that a sample of sufficient volume has been collected. In an embodiment of the disclosure, a combination of a non-diffusible dye and diffusible dye may be used together, such that the non-diffusible dye remains and provides an informative message when the diffusible dye disappears. For example, the diffusible dye may form the letters “in” in the word “insufficient” such that the non-diffusible dye remains and forms the word “sufficient” when a sufficient sample has been collected.

The sufficiency indicator may be a pH-sensitive substance that changes color when the sample is encountered. For example, multiple pH sensitive indicators responding to different pH values may be preset, such that a color change is observed whether the sample is acidic, basic, or neutral. According to an embodiment of the disclosure, a pH-changing substance, such as an acid or base, may be disposed within the absorbent material, such that the sample will be of the correct pH to elicit the desired color change in the sufficiency indicator.

A closure member may be used. The closure member is capable of sealing the open end of a sample receiving member when the fluid collector is inserted into the open end of a sample receiving member. For example, the closure member may be dimensioned to fit closely in the opening in the open end of the receiving member, and the closure member or the open end of the receiving member may include a compressible material, including without limitation natural rubber such as vulcanized rubber, synthetic rubber such as neoprene or nitrite rubber, plastic, ceramic, or any combination thereof, disposed at the interface between the closure member and the opening in the open end of the sample receiving member, that is capable of creating a seal, such as an airtight or a watertight seal, when the sample receiving member receives the fluid collector.

After the fluid collector has been inserted into the sample receiving member, a means for securing the fluid collector within the sample receiving member is contemplated. The means for securing may prevent removal of the fluid collector from the sample receiving member after it has been inserted therein. The means for securing the fluid collector within the sample receiving member may include at least one projection extending from the fluid collector that cooperates with the at least one projection located on the inner surface of the sample receiving member, where such projections may include for example at least one locking tab and/or at least one annular ring. According to an embodiment of the disclosure, a closure member on the fluid collector may form a sufficiently secure closure as to constitute means for securing the fluid collector within the sample receiving member.

The sample receiving member may also include a tamper-evident seal, such that attempting to tamper with the contents of the analyte testing device will result in a visual indicator, for example by tears or breakage visible in an imprinted seal, for example a tape or adhesive-backed foil having characters, symbols or a signature on a surface. Such a tamper-evident seal may be placed on the apparatus before its use, to create a visual confirmation that the intents of the apparatus have not been altered via the open end of the receiving member prior to testing, or after its use, to create a visual confirmation that the contents of the device have not been altered via the open end of the receiving member subsequent to testing. According to an embodiment of the disclosure, the means for securing the fluid collector within the sample receiving member may constitute a tamper evident seal, so that attempted removal of the fluid collector from the sample receiving member after it has been inserted therein may result in visible damage to the device.

According to an embodiment of the disclosure, the fluid collector includes a handle, made of for example wood, plastic, ceramic, or metal, and disposed, for example, at the end distal to the absorbent material. The handle may be removably attached, for example through an interference fit, adhesive, glue, or epoxy, that breaks or separates when the handle is twisted and/or pulled, or by a structure that allows the handle to be broken away, for example, a line of weakness.

The fluid collector may include a housing that at least partially surrounds the absorbent material. The housing may have multiple openings to allow the fluid sample to be absorbed by and expressed from the absorbent material. The openings in the housing may contain filtration members able to strain particulates from the fluid sample, resulting in reduction of the number of particulates that enter the absorbent material. The fluid collector may include a compression member able to compress the absorbent material against the housing. For example, the housing may be slideably coupled to a compression member with the absorbent material disposed between the compression member and an inner surface of the housing, such that the absorbent material may be compressed by movement of the compression member towards an inner surface of the housing. An embodiment of the disclosure includes means for securing the absorbent material in the compressed state, including without limitation cooperating threads, projections, and/or grooves operatively associated with the compression member and the housing. The absorbent material may be released from the compressed state before, concurrently with, or after encounter with the fluid sample, facilitating entry of the fluid sample into the absorbent material as the absorbent material returns to the relaxed state, creating suction. For example, the absorbent material may be operatively associated with a spring, such that compression of the absorbent material results in compression of the spring, and when compression is released the spring assists return of the absorbent material to the uncompressed state.

In some embodiments, the absorbent material is pre-soaked with the oropharyngeal lavage liquid prior to its insertion into the oral cavity. This facilitates entry of oropharyngeal lavage liquid into the oral cavity, for example when the subject is unconscious or unwilling to take the liquid into their mouth and gargle. The oropharyngeal lavage liquid can be, for example, water, a water-based solvent, a water-based solution, a pH buffer, or a non-alcoholic mouth rinse. The oropharyngeal liquid can also be designed to aid in increasing the solubility of drugs, drug metabolites, disease markers, and other substances in the oral cavity. In some embodiments, the oropharyngeal liquid is a water-based solvent for Therapeutic Drug Monitoring (TDM).

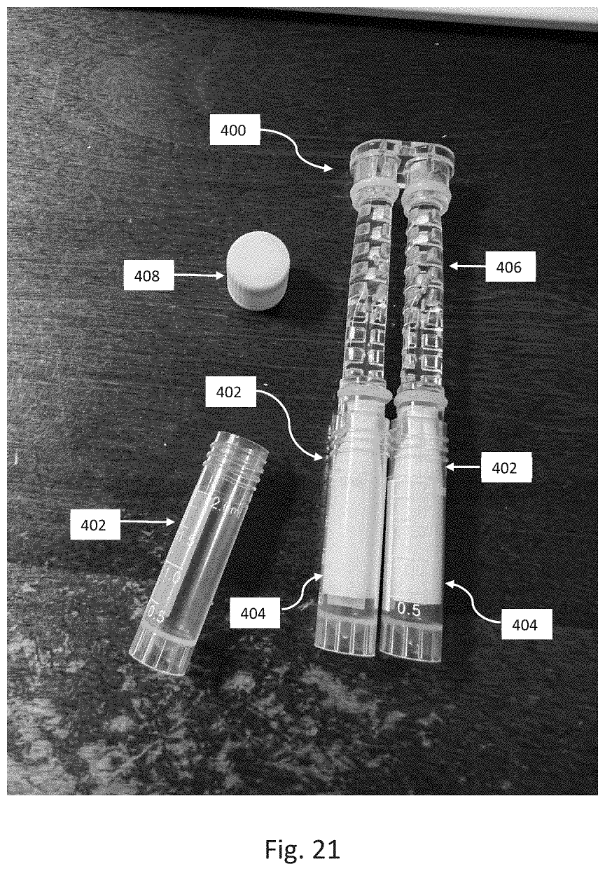

A representative example of this embodiment of the fluid collector, which is a dual-swab fluid collector 400, is depicted in FIG. 21, in which 402 is a collector housing member, both instances of 404 are the absorbent material in each prong, 406 is an upper segment, and 408 is a cap for the collector housing member, which can be used for storage of oropharyngeal lavage after collection. In this embodiment, the oropharyngeal lavage liquid is soaked into, and retained by, the absorbent material. Thus, in some embodiments, a fluid collector comprises: an upper segment; an absorbent material to absorb an oropharyngeal lavage liquid attached to one end of the upper segment; and a storage member for holding the absorbent material and the oropharyngeal lavage liquid; wherein the oropharyngeal lavage liquid is absorbed into the absorbent material and is inserted into the storage member. Advantageously, the use of this fluid collector streamlines the collection of analytes in oropharyngeal lavage liquids and makes it more efficient. For example, a separate container or packet of oropharyngeal lavage liquid is not needed.

In some embodiments, a method for collecting analyte from an oral cavity comprising: pre-soaking the absorbent material of the fluid collector of claim 28 with an oropharyngeal lavage liquid prior to insertion into the oral cavity; inserting the absorbent material of the fluid collector comprising the absorbent material and oropharyngeal lavage liquid into the oral cavity; ejecting the oropharyngeal lavage liquid into the oral cavity; absorbing the ejected oropharyngeal lavage liquid from the oral cavity; and removing the absorbent material of the fluid collector from the oral cavity.

In some embodiments of the method for analyte testing, the fluid collector comprises an absorbent material attached to a piston-like member operatively associated with a spring such that compression of the absorbent material results in compression of the spring, and when compression is released, the spring assists return of the absorbent material to the uncompressed state; wherein the oropharyngeal lavage liquid is ejected into the oral cavity by compression of the absorbent material and spring; and wherein the oropharyngeal liquid is absorbed from the oral cavity by release of compression of the absorbent material and spring.

In some embodiments of the method of collecting analyte, the analyte is a therapeutic drug or metabolite. Thus, the method can be used for therapeutic drug monitoring (TDM). In these embodiments, the oropharyngeal lavage liquid can be selected to dissolve the drug or drug metabolite to be monitored. Examples of therapeutic drugs that can be monitored by a method using this fluid collector include, for example, cardiac drugs, antibiotics, antiepilectics, bronchodilators, immunosuppressants, anti-cancer drugs, psychiatric drugs, metabolites thereof, and combinations comprising at least one of the foregoing. The cardiac drug can be, for example, digoxin, digitoxin, amiododarone, lidocaine, quinidine, procainamide, N-acetyl-procainamide (metabolite of procainamide), or a combination of at least one of the foregoing. The antibiotic can be an aminoglycoside, for example gentamicin, tobramycin, amikacin), vancomycin, chloramphenicol, or a combination comprising at least one of the foregoing. The antiepilectic can be, for example, phenobarbital, phenytoin, valproic acid, carbamazepine, ethosuximide, gabapentin, lamotrigine, levetiracetam, topiramate, zonisamide, eslicarbazepine acetate, felbamate, lacosamide, oxcarbazepine, pregabalin, rufinamide, stiripentol, tiagabine, vigabatrin, or a combination comprising at least one of the foregoing. The bronchodilator can be, for example, theophylline, caffeine, or a combination thereof. The immunosuppressant can be, for example, cyclosporine, tacrolimus, sirolimus, mycophenolate mofetil, azathioprine, or a combination comprising at least one of the foregoing. The anticancer drug can be methotrexate, any cytotoxic agent, or a combination comprising at least one of the foregoing. The psychiatric drug can be lithium, valproic acid, an antidepressant, for example imipramine, amitriptyline, nortriptyline, doxepin, or desipramine, or a combination comprising at least one of the foregoing. Thus, in some embodiments, the therapeutic drug comprises at least one of cardiac drugs, antibiotics, antiepilectics, bronchodilators, immunosuppressants, anti-cancer drugs, psychiatric drugs, and metabolites thereof.

A method of detecting SARS-CoV-2 virus antigen present in oropharyngeal lavage comprises the following steps: abstaining from placing liquid or food in an oral cavity of a user for a first period of time before collecting the oropharyngeal lavage; pre-soaking the absorbent end of a fluid collector with non-alcoholic mouth rinse prior to insertion into the oral cavity; inserting the absorbent end of the fluid collector comprising the absorbent material and non-alcoholic mouth rinse into the oral cavity to collect oropharyngeal lavage from the oral cavity, the oropharyngeal lavage including the non-alcoholic mouth rinse and particles retrieved from the oral cavity; removing the absorbent end of the fluid collector from the oral cavity; inserting the fluid collector into the sample receiving member of an analyte testing device for testing a fluid sample, with the absorbent end facing downward; and after a third period of time, reading a first indicator on a first test strip in the device to determine whether SARS-CoV-2 virus antigen is detected in the oropharyngeal lavage.

In some embodiments of this method of detecting SARS-CoV-2 virus, the absorbent material is operatively associated with a spring such that compression of the absorbent material results in compression of the spring, and when compression is released, the spring assists return of the absorbent material to the uncompressed state. Also in this method, the spring and absorbent material can be compressed by a piston-like member pushed inward in the direction of the absorbent material. Thus, someone administering the test can compress the piston-like member to compress the absorbent member and eject the non-alcoholic mouth wash or buffer solution into the oral cavity of the subject. The person administering the test can then release the piston-like member, so that the absorbent material is returned to its starting position, thereby creating suction so that the oropharyngeal lavage in the oral cavity is absorbed by the absorbent material. Thus, the method of detecting SARS-CoV-2 virus antigen further comprises compression of the piston-like member, causing the non-alcoholic mouth rinse to be ejected in the oral cavity, and release of compression on the piston-like member causes the spring and absorbent material to return to their original positions, thereby creating suction so that oropharyngeal lavage from the oral cavity is absorbed by the absorbent material.

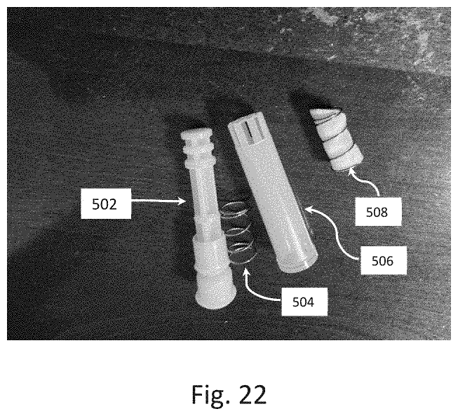

An embodiment of the fluid collector includes means for securing the absorbent material in the uncompressed state, including without limitation cooperating threads, projections, and/or grooves operatively associated with the compression member and the housing. Thus, in some embodiments, the fluid collector comprises an absorbent material attached to a piston-like member, wherein the absorbent material is operatively associated with a spring such that compression of the absorbent material results in compression of the spring, and when compression is released, the spring assists return of the absorbent material to the uncompressed state. Individual components of an embodiment of this fluid collector are depicted in FIG. 22, with piston-like member 502, spring 504, collector housing member 506, and absorbent material 508, shown inside of a spring. Thus, in some embodiments, a fluid collector comprises an absorbent material attached to a piston-like member, wherein the absorbent material is operatively associated with a spring such that compression of the absorbent material results in compression of the spring, and when compression is released, the spring assists return of the absorbent material to the uncompressed state. In an embodiment of the disclosure, the fluid collector is operatively associated with the lid of a fluid container including without limitation a urine cup. For example, the absorbent material may be disposed on the inner side of the lid, such that attachment of the lid to the fluid container results in contact between the absorbent material and a fluid sample. In certain embodiments of the disclosure, a portion of the fluid collector including the lid may be removably associated with a portion of the fluid collector including the absorbent material, allowing the absorbent material to be separated from the lid. The operative association of the fluid collector with the lid may include means for arresting the rotation of part of the fluid collector relative to the lid, including without limitation cooperating projections present on one member and grooves or slots present on the other member, for example to facilitate release of means by which the absorbent material is fixed in the compressed state.

### Saliva Producing Substances

Use of a saliva producing substance is contemplated by the present disclosure. Saliva producing substances elicit or increase saliva production in the test subject. For example, without limitation, the saliva producing substance may be sugars, salts, acids, or any combination thereof. In an embodiment of the disclosure, the saliva producing substance may be associated with a fluid collector, for example located on or in the absorbent material or the housing. In an embodiment of the disclosure, the saliva producing substance may be separated from the fluid collector, for example in the form of a gum, candy, or powder, for administration to the test subject before, during or after the fluid collector is inserted into the test subject's mouth.

For example, the sugar may be, without limitation, a monosaccharide, a disaccharide, a trisaccharide, an oligosaccharide, a polysaccharide, acarbose, allose, altrose, amylose, arabinose, cellobiose, cyclodextrin, alpha-cyclodextrin, beta-cyclodextrin, gamma-cyclodextrin, deoxyglucose, dextrin, dihydroxyacetone, erythrose, erythrulose, ficoll, fructo-oligosaccharides, fructose, galacto-oligosaccharides, galactose, gentiobiose, glucosamine, glucose, glyceraldehyde, glycogen, gulose, idose, inositol, inulin, isomaltose, lactose, lyxose, maltose, maltosyl-cyclodextrin, maltotriose, mannan-oligosaccharides, mannoheptulose, marinose, melexltose, mannitol, psiccae, raffinose, ribitol, ribose, ribulose, sedoheptulose, sorbitol, sorbose, sucrose, tagatose, talose, threose, trehalose, xylose, xylulose, or any combination thereof.

For example, without limitation, the salt may an inorganic salt, organic salt, acid salt, alkali salt, neutral salt, or amino acid salt, or any combination thereof. The salt may include a cation and an anion, for example without limitation thereto, the cation may be aluminum, ammonium, barium, beryllium, calcium, cesium, chromium(II), chromium(III), chromium(IV), cobalt(II), cobalt(III), copper(I), copper(II), copper(III), gallium, hydronium, iron(II), iron(III), lead(II), lead(IV), lithium, magnesium, manganese(II), manganese(III), manganese(IV), manganese(VII), nickel(II), nickel(III), nitronium, potassium, pyridinium, silver, sodium, strontium, tin(II), tin(IV), zinc, or any combination thereof, and the anion may be acetate, amide, tartrate, borate, bromate, bromide, carbonate, chlorate, chloride, chloride, chromate, citrate, cyanate, dichromate, dihydrogen phosphate, fluoride, formate, glutamate, hydride, hydrogen carbonate, hydrogen oxalate, hydrogen phosphate, hydrogen sulfate, hydrogen sulfite, hydroxide, hypobromite, hypochlorite, iodate, iodide, nitrate, nitride, nitrite, oxalate, oxide, perchlorate, permanganate, peroxide, phosphate, phosphide, phosphite, pyrophosphate, sulfate, sulfide, sulfite, telluride, thiocyanate, thiosulfate, or any combination thereof. For example, according to an embodiment of the disclosure, the salt may be sodium chloride or potassium chloride.

The acid may be any suitable acid known to a person skilled in the art, for example acetic acid, acrylic acid, adipic acid, alginic acid, an alkanesulfonic acid, an amino acid, ascorbic acid, benzoic acid, boric acid, butyric acid, carbonic acid, carboxylic acid, citric acid, a fatty acid, folic acid, formic acid, fumaric acid, gluconic acid, hydriodic acid, hydrobromic acid, hydrochloric acid, hydroquinosulfonic acid, isoascorbic acid, lactic acid, maleic acid, malic acid, malonic acid, methanesulfonic acid, nitric acid, oxalic acid, p-toluenesulfonic acid, para-bromophenylsulfonic acid, phosphoric acid, propionic acid, salicylic acid, stearic acid, succinic acid, sulfuric acid, tannic acid, tartaric acid, thioglycolic acid, toluenesulfonic acid, uric acid, or any combination thereof.

### Non-Alcoholic Mouth Rinse

The non-alcoholic mouth rinse can be a composition comprising: a therapeutic antimicrobial mouth rinse; a salt; and a mucolytic agent. Although the composition is characterized as a “non-alcoholic mouth rinse”, its use is not restricted to the oral cavity and oropharyngeal area. It can be generally useful for other medical and diagnostic purposes as well. Thus, “non-alcoholic mouth rinse” and “composition” are used interchangeably herein, and are distinguished from the “therapeutic antimicrobial mouth rinse”, which is a component of the “non-alcoholic mouth rinse”. In some embodiments, the composition is a stock solution comprising, based on the total volume of the composition: 12 to 62% w/v of the therapeutic antimicrobial mouth rinse; 5 to 25% w/v of the salt; and 2 to 8% w/v of the mucolytic agent. The composition can further comprise, based on the total volume of the composition, 15 to 81% w/v of other ingredients, for example, water, coloring, and flavoring. Thus, in some embodiments, the stock solution further comprises, based on the total volume of the composition, 15 to 81% w/v of at least one of water, a solvent, a flavoring, a sweetener, a coloring, a surfactant, a preservative, or a buffer. The mouth rinse is non-alcoholic, which means that it contains no alcohol, i.e. ethyl alcohol. In some embodiments, the non-alcoholic mouth rinse comprises less than 20, 10, 5, 1, or 0.1% w/v ethyl alcohol.

The stock solution can be diluted with distilled water in a volume ratio of stock solution to distilled water of, for example, 1:2, 1:5, or 1:10, to provide the non-alcoholic mouth rinse for end use in the methods and assays disclosed herein. For example, the non-alcoholic mouth rinse derived from a 1:2 dilution of the stock solution with distilled water can be used for seniors (age about 65 to about 99 years); the non-alcoholic mouth rinse derived from a 1:5 dilution of the stock solution with distilled water can be used for adults (age about 18 to about 64 years); and the non-alcoholic mouth rinse derived from a 1:10 dilution with distilled water can be used for youths (age about 2 to about 17 years). Thus in some embodiments, the non-alcoholic mouth rinse is obtained by diluting the stock solution with distilled water in a volume ratio of stock solution to distilled water of from about 1:2 to about 1:10. The amount of non-alcoholic mouth rinse suitable for use in the methods, assays, and kits disclosed herein can be about 0.5 to about 10 mL, and specifically about 1 to about 5 mL. In some embodiments, the amount of non-alcoholic mouth rinse is about 2 mL for each instance of the method, assay, or kit.

Surprisingly, the composition minimizes false positives in in vitro immunochromatographic assays utilizing the composition for collecting the liquid sample from the oral cavity and oropharyngeal area. The in vitro immunochromatographic assays for the qualitative detection of SARS-CoV-2 virus antigen utilizing the composition for collecting the liquid sample from the oral cavity and oropharyngeal area provide good agreement with RT-PCR test results, show good analytical sensitivity for different sources of the SARS-CoV-2 virus antigen, provide non-cross reactivity for a variety of bacterial and viral panes, and the test results are not interfered with by various medicinal and endogenous test substances.

The therapeutic antimicrobial mouth rinse component of the composition is an FDA-approved mouth rinse, i.e. a mouth rinse that kills the germs associated with bad breath and gingivitis. Thus, the mouth rinse is effective in disinfecting the sample and the oral cavity. For example, the mouth rinse can be at least 99%, 99.5%, 99.9%, or 99.99% effective in disinfecting the liquid sample. In some embodiments, the mouth rinse is at least 99.99% effective in disinfecting the liquid sample. Thus, in some embodiments of the composition, the therapeutic antimicrobial mouth rinse comprises an antimicrobial that is at least one of cetylpyridinium chloride (CPC), chlorhexidine (CHX), or at least one essential oil selected from the group consisting of eucalyptol, menthol, methyl salicylate, and thymol. In some embodiments of the composition, the therapeutic antimicrobial mouth rinse comprises, based on the total volume of the therapeutic microbial mouth rinse, a combination of 0.092% w/v eucalyptol, 0.042% w/v menthol, 0.060% w/v methyl salicylate, and 0.064% w/v thymol. The concentration of the therapeutic antimicrobial mouth rinse in the stock solution, based on the total volume of the stock solution, can be 12 to 62% w/v. The stock solution can be diluted with distilled water in a volume ratio of stock solution to distilled water of, for example, 1:2, 1:5, or 1:10, to provide the non-alcoholic mouth rinse for end use in the methods and assays disclosed herein.

In some embodiments, the salt can be an alkali metal or alkaline earth metal salt of a halide. Preferably, the salt comprises sodium chloride, to provide a saline solution. The concentration of the salt in the stock solution, based on the total volume of the stock solution, can be 5 to 25% w/v. The stock solution can be diluted with distilled water in a volume ratio of stock solution to distilled water of, for example, 1:2, 1:5, or 1:10, to provide the non-alcoholic mouth rinse for end use in the methods and assays disclosed herein.

In some embodiments of the composition, the mucolytic agent is at least one of guaifenesin (MUCINEX™), carbocysteine, erdosteine, mecysteine, bromhexine, hyperosmolar saline, mannitol powder, N-acetyl-L-cysteine (NAC), N-acetylcysteine, fudosteine, dornase alfa (PULMOZYME™), or thymosin β4. The concentration of the mucolytic agent in the stock solution, based on the total volume of the stock solution, can be 2 to 8% w/v. The stock solution can be diluted with distilled water in a volume ratio of stock solution to distilled water of, for example, 1:2, 1:5, or 1:10, to provide the non-alcoholic mouth rinse for end use in the methods and assays disclosed herein. In some embodiments of the composition, the therapeutic antimicrobial mouth rinse is at least one of cetylpyridinium chloride (CPC), chlorhexidine (CHX), or at least one essential oil selected from the group consisting of eucalyptol, menthol, methyl salicylate, and thymol; the salt comprises sodium chloride; and the mucolytic agent is at least one of guaifenesin (MUCINEX™), carbocysteine, erdosteine, mecysteine, bromhexine, hyperosmolar saline, mannitol powder, N-acetyl-L-cysteine (NAC), N-acetylcysteine, fudosteine, dornase alfa (PULMOZYME™), or thymosin β4.

### Fingerprint Identification

An embodiment of the present disclosure includes a fingerprint pad to provide identification of an individual associated with the test, such as the test subject, test administrator, and/or one or more witnesses. The fingerprint pad may employ any suitable fingerprinting methodology, for example, without limitation, ink-based, immunoassay-based, electronic, semi-inkless, or inkless. In an embodiment of the disclosure, the fingerprint pad may be able to collect multiple fingerprints. For example, the analyte testing device may have multiple fingerprint pads, one fingerprint pad of sufficient size to accommodate multiple fingerprints, or an electronic fingerprint pad.

The fingerprint pad may be an ink-based fingerprint pad. An embodiment of the disclosure includes a dispenser able to dispense an ink that can elicit a signal in the ink-based fingerprint pad. The fingerprint pad may also be inkless or semi-inkless, for example requiring no ink or compatible with an activator that appears transparent on the subject's skin, is readily cleaned off the subject's skin, or readily disappears, for example, when the subject's hands are rubbed together. According to an embodiment of the disclosure, the inkless fingerprint pad may be immunoassay-based, for example as described in U.S. Pat. No. 6,352,663 to Raouf A. Guirguis, issued Mar. 5, 2002 (the “'863 patent”), and U.S. Pat. No. 5,244,815 to Raouf A. Guirguis, issued Sep. 14, 1993 (the “'815 patent”), which are incorporated herein by reference in their entirety. The immunoassay-based fingerprint pad may or may not be in fluid communication with a sample receiving member. Other embodiments of the disclosure may incorporate various features of the embodiments disclosed in the '863 and '815 patents. In an embodiment of the disclosure having an inkless or semi-inkless fingerprint pad that requires an activator to elicit a signal, the analyte testing device may also include a dispenser to dispense the activator. According to an embodiment of the disclosure, the fingerprint pad may have a surface, such as an absorbent or adhesive surface, able to gather sweat, oils, and/or skin cells when a finger is pressed against it, that may require further processing to permit clear visualization of the fingerprint.

According to an embodiment of the disclosure, an inkless fingerprint pad may be an electronic fingerprint pad, including without limitation an optical scan fingerprint reader or a solid-state fingerprint reader. An embodiment of the disclosure includes a memory element, including without limitation volatile or non-volatile memory, for example a hard disk, floppy disk, magnetic tape, optical disk, flash memory, holographic memory, EEPROM, RAM, DRAM, SDRAM, or SRAM coupled to the fingerprint pad for storage of one or more fingerprints. According to an embodiment of the disclosure, the electronic fingerprint pads may have electrically charged surface elements, wherein portions of the surface are electrically discharged upon contact with the finger surface, such as the ridges of the finger surface, such that the fingerprint is recorded in the pattern of discharged elements, whereby the fingerprint pattern may be stably stored within the surface for a time after it is created until it is read, for example through connection of the apparatus with an external device, including without limitation a base station. An embodiment of the disclosure includes means of transmission of the captured fingerprint, for example to an external device or network, including without limitation through a hard-wired connection, for example employing wires, cables, or a docking station or docking connector, for employing a connection including without limitation USB, IEEE 1394, serial, parallel, SCSI, or a wireless connection, for example employing infrared, RF, IEEE 802.11, Bluetooth, IEEE 802.15, or Wi-Fi.

In an embodiment of the disclosure, a cover encloses the fingerprint acquisition pad. The cover may be secured using various mechanisms, for example, without limitation, a tab-and-slot connector, latch, spring latch, adhesive tape, or security tape. The cover may be secured prior to fingerprint acquisition and/or after fingerprint acquisition.

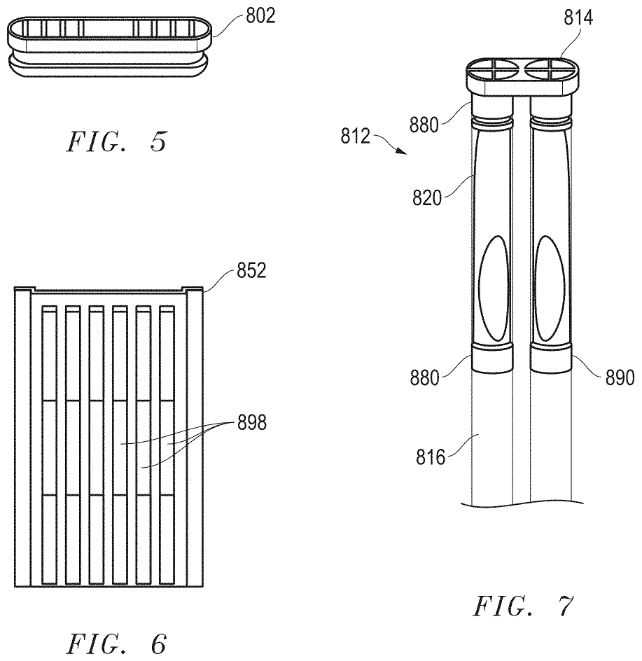

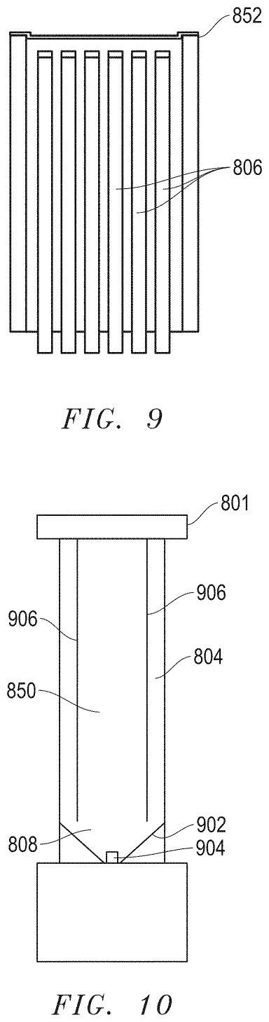

Referring to FIG. 1-10, a fluid collection and analyte testing device 800 according to embodiments of the disclosure are shown. In one embodiment, an analyte testing device 800 includes a fluid collector 812, to collect a fluid sample from a test subject, and a housing 801 to test and retain the fluid sample. The fluid collector 812 may include single or multiple collectors for the collection of the fluid sample. In one embodiment of the disclosure, a dual-swab split sample testing device, the fluid collector 812 includes a dual collector or two-prong collector with prongs that are substantially identical. However, a wide variety of modifications to the prongs may be implemented without detracting from the spirit of the disclosure, including but not limited to, prongs of varying size, shape and materials. Each prong of the fluid collector 812 includes an upper segment 820 having an upper surface, a closure member 814, sealing members 880, a compression member 890, and a collector 816 made from an absorbent material. A wide variety of absorbent materials capable of acquiring and storing a fluid sample may be used without detracting from the spirit of the disclosure, including but not limited to a swab, a sponge, and a material that dissolves subsequent to collection of the sample. In one embodiment of the disclosure, the absorbent material may be saturated with a saliva-producing substance to aid in the collection of the fluid sample. Additionally, the collector 816 may include a sufficiency or visual indicator to indicate when a sufficient amount of the fluid sample is present in the collector 816. U.S. Pat. No. 9,198,641 entitled “Specimen Sample Collection System” describes one prior art sufficiency or visual indicator system and is hereby incorporated by reference. The fluid collector 812 receives a fluid sample from a test subject and temporarily stores the fluid sample until it is transferred to the housing 801. In one embodiment of the disclosure, the fluid collector 812 receives a fluid sample from a test subject to be used in a split sample fluid testing device.

One advantage of the embodiments disclosed herein is the limited amount of test material needed for testing and retention. According to one embodiment of the disclosure, only four hundred eighty (480) microliters are required for the successful analysis of twelve (12) test strips, forty (40) microliters for each test strip and only seven hundred (700) microliters are required from each prong. The minimum retention amount necessary is approximately the same volume as needed for the test strips. This quantity of a sample is obtainable through the fluid collector described herein. For example, forty (40) by ten (10) millimeter cylindrical collectors 816 collected a fluid, as the test sample, in amounts set forth in the tables below.

TABLE 1
Single-Prong Sample Collection Amount

| | Total Fluid | Fluid in | | |
|---|---|---|---|---|
| | Amount | Chamber after | | Collection |
| | Collected | Extraction | Fluid Loss | Time |
| Sample | (ml) | (ml) | (ml) | (seconds) |
| 1 | 1.7 | 1.23 | 0.47 | 67 |
| 2 | 1.5 | 1.13 | 0.37 | 89 |
| 3 | 1.66 | 1.31 | 0.35 | 73 |
| 4 | 1.48 | 1.11 | 0.37 | 105 |
| 5 | 1.36 | 1.04 | 0.32 | 114 |
| 6 | 1.71 | 1.29 | 0.42 | 101 |
| 7 | 1.65 | 1.33 | 0.32 | 113 |
| 8 | 1.36 | 1.03 | 0.33 | 97 |
| Average | 1.55 | 1.18 | 0.37 | 95 |

TABLE 2
Dual-Prong Sample Collection Amount

| | | Fluid in 1^{st} | Fluid in 2^{nd} | | |
|---|---|---|---|---|---|
| | Total Fluid | Chamber | Chamber | | |
| | Amount | after | after | | Collection |
| | Collected | Extraction | Extraction | Fluid Loss | Time |
| Sample | (ml) | (ml) | (ml) | (ml) | (seconds) |
| 1 | 3.67 | 1.01 | 1.34 | 1.32 | 106 |
| 2 | 3.07 | 1.07 | 1.08 | 0.92 | 128 |
| 3 | 3.23 | 1.22 | 1.10 | 0.91 | 145 |
| 4 | 2.92 | 1.17 | 0.98 | 0.77 | 183 |
| 5 | 3.10 | 1.22 | 1.19 | 0.69 | 124 |
| 6 | 2.78 | 1.14 | 1.05 | 0.59 | 152 |
| 7 | 3.11 | 1.10 | 1.10 | 0.91 | 181 |
| 8 | 2.96 | 1.12 | 1.08 | 0.76 | 133 |
| Average | 3.11 | 1.13 | 1.12 | 0.86 | 144 |

As shown in Tables 1 and 2, the fluid collector 816, according to an embodiment of the disclosure, on average collects an amount of fluid samples in excess of the amounts required for testing and/or retention in approximately ninety (90)-one hundred fifty (150) seconds. A wide range of collection amounts are anticipated, depending upon the testing requirements, without detracting from the spirit of the disclosure.

The housing 801 includes a sample receiving member 818 to receive the fluid collector 812. In one embodiment of the disclosure, the sample receiving member 818 includes two collection chambers 874 to receive the two-pronged fluid collector 812 through two openings 870, thus forming a split sample. The sample receiving member 818 is in fluid communication with a fluid sample retention member, such as a confirmation collection chamber 810, and a test cartridge member 804. The test cartridge member 804 includes a test cartridge chamber 850; a test cartridge 852; at least one membrane test strip 806 located on the test cartridge 852 to indicate the presence or absence of at least one analyte; and a test cartridge fluid reservoir 808 in fluid communication with the test cartridge 852 and the sample receiving member 818. In one embodiment of the disclosure, the housing 801 includes windows on the front, back or both sides of the housing 801 for viewing of the membrane test strip 806. A wide variety of housings 801 may be implemented without detracting from the spirit of the disclosure, including but not limited to forming the housing 801 from a clear material allowing the membrane test strips 806 to be viewed without a window. The test cartridge fluid reservoir 808 may be formed in a variety of shapes without detracting from the spirit of the disclosure, including a v-shaped chamber with a flat bottom 902 as shown in FIG. 10. The test cartridge fluid reservoir 808 with a flat bottom v-shaped chamber 902 allows the test cartridge 852 and membrane test strips 806 to fully engage the fluid sample while maintaining a small volume of the fluid sample. In one embodiment of the disclosure, the v-shaped chamber with a flat bottom 902 forms a volume of less than seven hundred (700) microliters. An opening 904 at the bottom of the second collection chamber 874 is in fluid connection via a channel 910 with the test cartridge fluid reservoir 808. A wide variety of connection mechanisms may be implemented to connect the second collection chamber 874 and the test cartridge fluid reservoir 808 without detracting from the spirit of the disclosure, including but not limited to, tubes, piping, channels molded or carved into the housing 801, or any other suitable structure.

In one embodiment of the disclosure, the sample receiving member 818 includes a first collection chamber 874 in fluid communication with the confirmation collection chamber 810 and a second collection chamber 874 in fluid communication with the test cartridge member 804. The first collection chamber 874 and the confirmation collection chamber 810 are not in fluid communication with any other elements or components of the housing 801, including the second collection chamber 874 and the test cartridge member 804. The second collection chamber 874 is in fluid communication with the test cartridge fluid reservoir 808, which is in fluid communication with the test cartridge 852 and the membrane test strips 806.

Once the fluid collector 812 receives a fluid sample from a test subject, the fluid collector 812 is inserted into the two collection chambers 874 in the sample receiving member 818, through two openings 870. The fluid sample is expelled by compressing the collector 816 between the compression member 890 of both prongs against the bottom surface of the lower portion of the two collection chambers 874, thereby releasing the entrapped fluid into the housing 801. The fluid sample from the test subject is delivered from the first collection chamber 874 to the confirmation collection chamber 810 and from the second collection chamber 874 to the test cartridge fluid reservoir 808. The fluid sample is only obtained a single time with the multiple or two-prong fluid collector 812 while maintaining fluid sample integrity through the collection, storage and analysis of the fluid sample with two distinct storage areas: the confirmation collection chamber 810 and the test cartridge fluid reservoir 808. Once the fluid collector 812 is secured within the housing 801, the fluid sample from the confirmation collection chamber 810 is not in fluid communication with the fluid sample in the test cartridge fluid reservoir 808. The confirmation collection chamber 810 fluid sample may be accessed by a third party as previously disclosed, typically subsequent to the testing of the fluid sample in the test cartridge fluid reservoir 808. In one embodiment of the disclosure, the confirmation collection chamber 810 is located below the first collection chamber 874. In another embodiment of the disclosure, the confirmation collection chamber is removable from the housing 801 after the fluid sample is extracted from the collector 812. The fluid collector 812 secures the fluid sample within the sample receiving member 818 with the sealing members 880 to form a seal between the fluid collector and the fluid collection chambers 874. In one embodiment, each prong of the fluid collector 812 includes sealing members 880 located near the top and bottom of the upper segment 820 to seal both of the two collection chambers 874. The sealing members 880 include sealing rings. The sealing rings may be attached at locations close to the top and bottom of the upper segment 820. Generally, the dimensions of sealing members 880, and the sealing rings, comport with the interior dimension of the two collection chambers 874 in order to prevent the sample from escaping through the openings 870. Additionally, the fluid collector 812 is secured by the locking closure member 814. In one embodiment of the disclosure, the locking closure member 814 includes at least one projection extending from the fluid collector 812 that cooperates with the at least one projection located on the inner surface of the sample receiving member 818, where such projections may include for example at least one locking tab and/or at least one annular ring. According to one embodiment of the disclosure, a closure member on the fluid collector 812 may form a sufficiently secure closure as to constitute means for securing the fluid collector 812 within the sample receiving member 818.

The test cartridge 852 includes slots for one or more membrane test strips 806. In one embodiment of the disclosure, the test cartridge 852 includes locations or slots for membrane test strips 806 on both the front and back of the test cartridge 852 in a back-to-back formation. The test cartridge 852 may include multiple locations for the membrane test strips 806 on either the front, back or both sides of the test cartridge 852. The test cartridge 852 may allow for a wide number of membrane test strips 806 to be attached to the test cartridge without detracting from the spirit of the disclosure, including but not limited to, six (6), twelve (12), or twenty-four (24) membrane test strips 806. A wide variety of attachment mechanism may be used to attach the membrane test strips 806 to the test cartridge 852 without detracting to the spirit of the disclosure, including but not limited to, slotted membrane test strip holders 898 on the test cartridge 852 and a protective sheet attached to the test cartridge 852 that covers and impedes movement of the test strip 806 from the test strip holders 898. After the membrane test strips 806 are attached to the test cartridge 852, the test cartridge 852 is inserted into the test cartridge chamber 850 through a test cartridge chamber opening 872 and is placed between test cartridge guides 906 on both ends of the test cartridge chamber 850. In one embodiment of the disclosure, the membrane test strips 806 extend beyond the test cartridge 852 into the test cartridge fluid reservoir 808. A test cartridge cap 802 is inserted into the test cartridge chamber 850 to secure the test cartridge 852 within the housing 801. In one embodiment of the disclosure, the test cartridge cap 802 is fixedly attached to the test cartridge 852 prior to insertion into the test cartridge chamber 850 or the test cartridge cap 802 and the test cartridge 852 are formed from a continuous material. In another embodiment of the disclosure, the test cartridge cap 802 attaches to a top edge of the test cartridge chamber opening 872. Advantageously, different versions of the test cartridge 852 may be developed to test different combinations of analytes, thereby allowing the test administrator to select the appropriate analyte test suite at the test site. The test cartridge chamber 850, the test cartridge cap 802, or a combination of both may include a locking mechanism known to those skilled in the art to secure the test cartridge 852 within the test cartridge chamber 850, thereby preventing the removal of the test cartridge 852 from housing 801.

In one embodiment of the disclosure, the test strips 806 may indicate genomic or proteomic information in that certain DNA sequences or proteins may be detected that are genetic predispositions for certain diseases such as various forms of cancer, diabetes, etc.

The test cartridge 852 and test cartridge cap 802 may be made from a variety of materials without detracting from the spirit of the disclosure, including but not limited to, plastic, ceramic, metal, glass, wood, rubber, polymer, fiber-reinforced polymer, or any combination thereof. In one embodiment, the test cartridge 852 is formed from plastic and is approximately 70 millimeters in height, 40 millimeters in width, and 5 millimeters in thickness.

After the fluid sample has been expelled from the fluid collector 812 into the test cartridge fluid reservoir 808, the fluid sample encounters the proximal end of the membrane test strip 806 and begins to move upward towards the upper end of the membrane test strip 806 by capillary action. Each membrane test strip 806 generally indicates the presence or absence of at least one analyte. A single drug, or class of drugs, is indicated by each membrane test strip 806, including without limitation, for example, cocaine (COC), amphetamine (AMP), methamphetamine (mAMP), marijuana (THC), methadone (MTD), phencyclidine (PCP), morphine, barbiturates, benzodiazepines, or alcohol. In one embodiment, the test strips 806 may be lateral flow test strips.

In one embodiment, the test cartridge chamber 850 and/or the test cartridge fluid reservoir 808 may be attached to an electrical device that supplies an electric current to the fluid sample. The electrical current may be used to separate elements within the fluid sample prior to testing of the fluid sample.

The housing may include an immunoassay-based fingerprint acquisition pad 860 to positively identify an individual associated with the fluid collection and analyte test. In one embodiment of the disclosure, the second collection chamber and/or the test cartridge fluid reservoir 808 may be in fluid communication with the immunoassay-based fingerprint acquisition pad 860. The immunoassay-based fingerprint acquisition pad 860 may be removably connected to the housing 801 or fixedly attached to the housing 801. The immunoassay-based fingerprint acquisition pad 860 is enclosed by a cover 866 that is held closed by closure member 864 and pivots into the opened position on the axis defined by the hinges 862. The cover 866 may be secured after the fingerprint of the test subject has been acquired, using various locking mechanisms, including without limitation a tab-and-slot arrangement, or security tape.

The immunoassay-based fingerprint acquisition pad 860 may be a stand-alone device connected to the housing 801 or the immunoassay-based fingerprint acquisition pad 860 may be in fluid communication with the test cartridge fluid reservoir 808. The immunoassay-based fingerprint acquisition pad 860 in fluid communication with the test cartridge fluid reservoir 808 functions as previously disclosed.

In an embodiment of the disclosure, the immunoassay-based fingerprint acquisition pad 860 includes a compressible, porous reaction medium, having a control zone and a plurality of reaction zones, arranged on a porous support. The control zone includes a control reagent to identify the fluid sample donor, and each reaction zone includes a reaction reagent to determine the presence of a specific analyte in the fluid sample. The control reagent includes a member of a predetermined ligand/receptor binding pair. Similarly, each reaction reagent includes a member of a predetermined ligand/receptor binding pair. Various ligand/receptor binding pairs for use within the control and reaction zones are discussed in the '863 and '815 patents.

In an embodiment of the disclosure, the immunoassay-based fingerprint acquisition pad 860 is fluidly coupled to the collection chamber 874. A signal-producing agent, located on upper surface of the porous support or the lower surface of the reaction medium, mixes with the fluid sample provided to the immunoassay-based fingerprint acquisition pad 860. The production of an image or pattern which identifies the person providing the sample is accomplished by applying a fingertip to the upper surface of the reaction medium and compressing the reaction medium so that the fluid sample/signal-producing agent mixture permeates the reaction medium, allowing the control zone ligand/receptor reaction to take place so that the members of this immunological pair bond with the signal-producing agent to produce the fingerprint image. Similarly, the presence or absence of a specific analyte in the fluid sample is indicated within each reaction zone by the reaction of each specific reaction reagent with the fluid sample/signal-producing agent mixture.

### Method of Detecting SARS-CoV-2

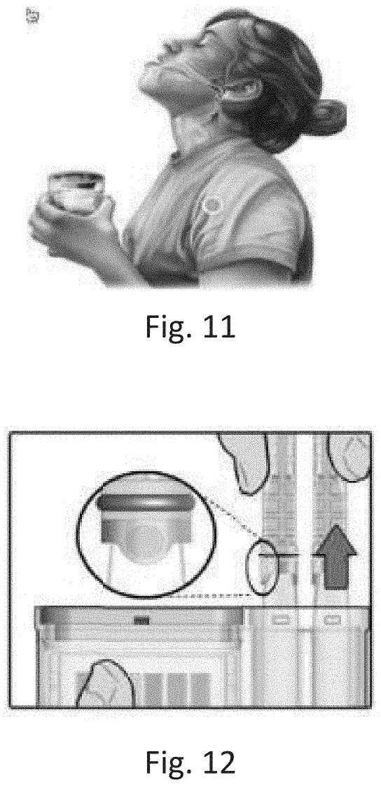

The present disclosure also relates to a method of detecting SARS-CoV-2 virus antigen present in an oropharyngeal lavage. The method comprises abstaining from placing liquid or food in an oral cavity of a user for a first period of time before collecting the oropharyngeal lavage. For example, the user should abstain from all liquids and foods for at least fifteen minutes or twenty minutes before performing the test. Test kit components, including the non-alcoholic mouth rinse, fluid collector, and apparatus 800 should be brought to room temperature before conducting the test. The method further comprises pouring a non-alcoholic mouth rinse into the oral cavity, and swishing and gargling the non-alcoholic mouth rinse for a second period of time to distribute the non-alcoholic mouth rinse around the inside of the oral cavity and throughout an oropharyngeal space of the user. The second period of time can be, for example, at least 30 seconds, or at least one minute. The mouth rinse should not be spit out of the user's mouth or swallowed, but gargled. Gargling is done by the user tilting their head back and gently blowing air through the liquid in the back of the oropharyngeal space. This step is illustrated in FIG. 11.

The non-alcoholic mouth rinse that is used for testing can be, for example, OroPharyngeal Lavage™ (OPL™). Advantageously, the non-alcoholic mouth rinse breaks up the bacterial and viral contents of the user's mouth, thereby releasing biological analytes that can be detected by the test, for example bacterial and viral antigens. The bacterium or virus can be, for example, SARS-CoV-2 virus, Herpes simplex virus, Epstein-Barr virus, cytomegalovirus, Moraxella catarrhalis, Norocardia sp., Streptococcus mutans, Streptococcus oralis, Eikenella sp., Neisseria sp., and Streptococcus salivarius. In addition to being used to break up virus-containing particles from a user's mouth, OPL™ is able to disinfect the sample. Test results have shown that 99.99% of virus samples are disinfected by OPL™. Advantageously, this reduces the spread of the SARS-CoV-2 infection between individuals during testing procedures. The non-alcoholic mouth rinse is described in more detail above.

The testing of oral cavity fluids (saliva and oral mucosa) is a more effective method for detecting the presence of at least one SARS-CoV-2 and SARS-CoV-2 variant (Omicron variant) than testing of nasal swabs. In particular, oropharyngeal lavage sampling is more sensitive and effective than nasal sampling. In testing for the presence of SARS-CoV-2 Omicron variant, saliva and oropharyngeal lavage using the non-alcoholic mouth rinse is effective, because the SARS-CoV-2 Omicron variant has been shown more likely to reside in the oral cavity and colonize the oral cavity than the nasal cavity. Thus, it is advantageous to use the non-alcoholic mouth rinse to collect samples from the oral cavity. Furthermore, it is beneficial to have a testing method such as the one disclosed herein, because of the ability of the non-alcoholic mouth rinse to disinfect the oral cavity and thereby reduce the amount of particles containing SARS-CoV-2 Omicron variant in the oral cavity, and to help prevent the spread of SARS-CoV-2 Omicron variant. In addition to SARS-CoV-2 and SARS-CoV-2 variants, the analyte can also be a variety of markers derived from Herpes simplex virus, Epstein-Barr virus, cytomegalovirus, Moraxella catarrhalis, Norocardia sp., Streptococcus mutans, Streptococcus oralis, Eikenella sp., Neisseria sp., and Streptococcus salivarius.

As discussed above, the non-alcoholic mouth rinse breaks up microbiota in a user's mouth, e.g. viruses and bacteria, and interacts with saliva in order to activate contents throughout the inside of the mouth, including the inside of the cheeks, the tongue and back of the throat. The non-alcoholic mouth rinse can be any non-alcoholic mouth rinse capable of breaking up the microbiota in a user's mouth, i.e. capable of breaking up viral and bacterial components of a user's mouth, in order to detect the presence of various markers therein. In some embodiments, the non-alcoholic mouth wash is one as described herein. The bacterium or virus can be, for example, SARS-CoV-2 virus, Herpes simplex virus, Epstein-Barr virus, cytomegalovirus, Moraxella catarrhalis, Norocardia sp., Streptococcus mutans, Streptococcus oralis, Eikenella sp., Neisseria sp., and Streptococcus salivarius.

The method further comprises inserting an absorbent end of a fluid collector comprising an absorbent material into the oral cavity to collect the oropharyngeal lavage from the oral cavity, the oropharyngeal lavage including the non-alcoholic mouth rinse and particles retrieved from the non-alcoholic mouth rinse. In particular, the absorbent end of the fluid collector is inserted under the tongue of the user and held in place for a fourth period of time, and wherein the absorbent end of the fluid collector is configured to soak up the entire oropharyngeal lavage of the oral cavity. The fourth period of time can be at least one minute.

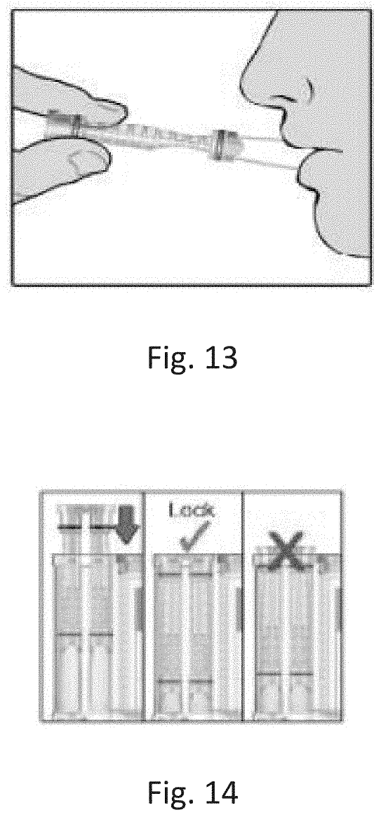

Specifically, an absorbent end disposed at the first end of the fluid collector is inserted into the user's mouth, underneath the tongue, and held in position for a third time period, such as one minute. The fluid collector has an absorbent tab or surface configured for collecting oropharyngeal lavage disposed at the first end of the fluid collector. The absorbent end is configured to be inserted into the oral cavity of the user and to absorb at least the non-alcoholic mouth rinse and contents of the oral cavity of the user, i.e. the oropharyngeal lavage obtained from the non-alcoholic mouth rinse. The user holds the fluid collector in place for the fourth period of time without biting, chewing, or moving the absorbent end. The absorbent end soaks the liquid contents from the user's mouth, i.e. the oropharyngeal lavage, which also includes the particles, for example viral and bacterial antigen particles extracted from the oral cavity with the non-alcoholic mouth rinse. FIG. 13 illustrates the placement of the absorbent end of a single-swab fluid collector under the user's tongue.

In some embodiments, the user also scrubs the inside of the mouth, including the cheek, gums, and tongue, with the absorbent end of the fluid collector, before inserting it under the tongue. The scrubbing is done without biting, sucking, or chewing on the absorbent end. The scrubbing is also done without spitting out or swallowing the non-alcoholic mouth rinse (oropharyngeal lavage), so that it can be absorbed by the absorbent end of the fluid collector for testing.

In the analyte testing device 800, the fluid collector 812 can be conveniently positioned in the sample receiving member 818. The end of the fluid collector opposite the absorbent end can be covered with a protective cap, locking closure member 814, as depicted in FIGS. 1-3 and 7. The protective cap is designed to keep the absorbent end of the fluid collector sterile when stored in the sample receiving member until removal from the sample receiving member and insertion into the user's mouth. When the fluid collector is positioned in the sample receiving member, it is easily pulled out of the sample receiving member for use. Prior to doing so, the protective cap, locking closure member 814, when present, must be removed from the sample receiving member 818. See FIG. 12 for an illustration of removal of the fluid collector from the sample receiving member of the analyte testing device.

The method further comprises removing the absorbent end of the fluid collector from the oral cavity. After the fourth period of time for absorbing the contents from the user's mouth into the absorbent end, e.g. at least one minute, the fluid collector is then removed from the oral cavity. After removal of the absorbent end of the fluid collector from the oral cavity, the user may safely swallow or spit out any remaining oropharyngeal lavage in their mouth that was not absorbed by the absorbent end of the fluid collector.

The method further comprises inserting the fluid collector into the sample receiving member of an analyte testing device for testing a fluid sample, with the absorbent end facing downward. In particular, the method can further comprise insertion of the fluid collector into the sample receiving member, absorbent end facing downward, until the fluid collector locks into place and the end of the fluid collector opposite the absorbent end is flush with the top of the sample receiving member. This step is illustrated for a dual-swab fluid collector in FIG. 14, in which the left column illustrates the fluid collector partially inserted into the sample receiving member and the direction of insertion; the center column illustrates the fluid collector locked into place in the sample receiving member; the right column illustrates the fluid collector not fully inserted into the sample receiving member.

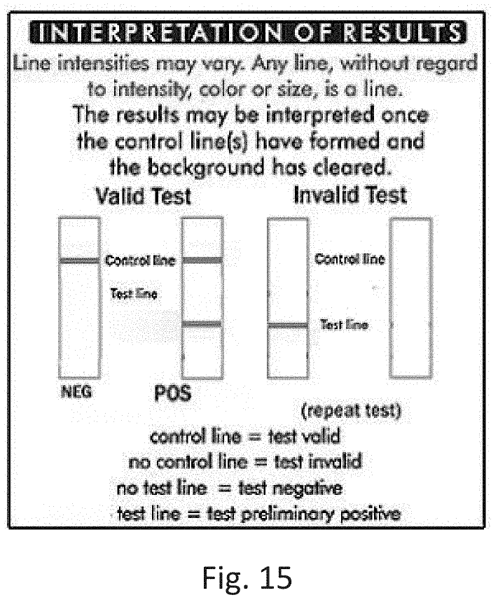

The method further comprises, after a third period of time, reading a first indicator on a first test strip in the apparatus to determine whether SARS-CoV-2 virus antigen is detected in the oropharyngeal lavage. The third period of time allows for the oropharyngeal lavage to migrate from the absorbent end of the fluid collector into the test cartridge fluid reservoir, to the bottom of the test strip, and from the bottom to the top of the test strip, and also allows time for the background appearance of the test strip to clear of any residual color. Thus, the third period of time can be 7 to 15 minutes, for example 7 to 10 minutes. Once the test is completed, an indicator on the test strip displays the results for the analyte (e.g., SARS-CoV-2 antigen or SARS-CoV-2 antibodies). The test strip confirms whether the analyte was detected in the oropharyngeal lavage collected from the user or not. Interpretation of the test results is illustrated in FIG. 15. The test strips are configured with two horizontal zones positioned at about ⅓ and about ⅔ of the length of the test strips. The lower zone is a test zone, and the upper zone is a control zone. A horizontal line in the lower zone indicates a positive test for the analyte, and a horizontal line in the lower zone indicates a valid test. The line intensities may vary. Any line visible to the naked eye, without regard to its color, intensity, or size is considered a positive test. The left test strip in FIG. 15 shows no test line, indicating a negative test, and a control line, indicating a valid test. The second to left test strip in FIG. 15 shows a test line, indicating a positive test, and a control line, indicating a valid test. The second to right test strip in FIG. 15 shows a test line, indicating a positive test, and no control line, indicating an invalid test. The right test strip shows no test line, indicating a negative test, and no control line, indicating an invalid test.

The analyte testing device used in the method comprises: a sample receiving member having an opening for receiving the oropharyngeal lavage, wherein the sample receiving member includes at least first and second sample collection chambers, and a sample retention member in fluid communication with the first sample collection chamber to retain a portion of the oropharyngeal lavage; and a test cartridge member in fluid communication with only the second sample collection chamber to indicate the presence or absence of SARS-CoV-2 virus antigen in the oropharyngeal lavage; wherein the test cartridge member comprises a test cartridge having a front set of test strip slots and a back set of test strip slots, and at least one test strip to indicate the presence or absence of SARS-CoV-2 virus antigen in the oropharyngeal lavage. The analyte testing device, its various components, and the functions of the components, are discussed in greater detail elsewhere herein.

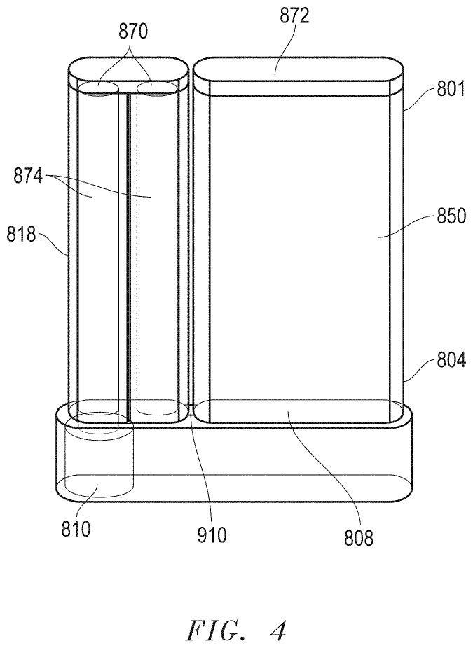

For example, and with reference to FIGS. 1 and 4, the test cartridge member 804 further comprises a test cartridge chamber 850 and a test cartridge fluid reservoir 808 in fluid communication with the sample receiving member 818, wherein the test cartridge 852 is inserted into the test cartridge chamber and wherein the at least one test strip 806 is in fluid communication with the test cartridge fluid reservoir. The test cartridge fluid reservoir can be a V-shaped chamber with a flat bottom. In some embodiments, the V-shaped chamber with a flat bottom forms a volume of less than or equal to four hundred eighty (480) microliters. In some embodiments, the test cartridge member further comprises a test cartridge cap 802 to secure the test cartridge 852.

The test cartridge comprises a plurality of test strip slots, which can accommodate the same number of test strips. For example, the front set of test strip slots can comprise at least six test strip slots and the back set of test strip slots can comprise at least six test strip slots. A plurality of test strips can be inserted into the front set of test strip slots and the back set of test strip slots, and the plurality of test strips are in fluid communication with the test cartridge fluid reservoir 808. Advantageously, this design feature enables testing for a plurality of analytes, including a variety of viral and bacterial antigens, and antibodies. Thus, the method can further comprise detecting SARS-CoV-2 virus IgM/IgG antibodies, wherein the test cartridge has a second test strip to indicate the presence or absence of SARS-CoV-2 virus IgM/IgG antibodies.

The analyte testing device can be a single-swab analyte testing device, and embodiment of which is illustrated in FIG. 17. This device includes a single-swab fluid collector 601 having absorbent material 602, a test cartridge 604, and analyte testing device housing 603 configured to receive the test cartridge 604 with accompanying test strips and single-swab fluid collector 601. Thus, in some embodiments, the analyte testing device is a single-swab analyte testing device and the fluid collector is a single-swab fluid collector having a single prong of absorbent material, wherein the single-swab analyte testing device is configured to receive the single-swab fluid collector.

Advantageously, the analyte testing device can also be a multi-swab testing device and the fluid collector can be a multiple-swab fluid collector having multiple prongs of absorbent material, wherein the multi-swab testing device is configured to receive the multiple-swab fluid collector. For example, the analyte testing device can be a dual-swab analyte testing device and the fluid collector can be a dual-swab fluid collector having two prongs of absorbent material, wherein the dual-swab analyte testing device is configured to receive the dual-swab fluid collector. This dual-swab fluid collector is also referred to as a split sample (dual sample) fluid collector herein. This design has the advantage of providing two oropharyngeal samples, one for detection of an analyte, for example SARS-CoV-2 virus antigen, and another for sample retention. As mentioned above, the retained portion of the oropharyngeal lavage can be used for further testing, for example for confirmation of a test result obtained using a test strip, to test for the presence or absence of other analytes, and/or for confirmation of the test subject's identity. In connection with the dual-swab fluid collector, and with reference to FIG. 4, the first and second sample collection chambers 874 of the analyte testing device are configured to receive a dual-swab fluid collector having two prongs of absorbent material.

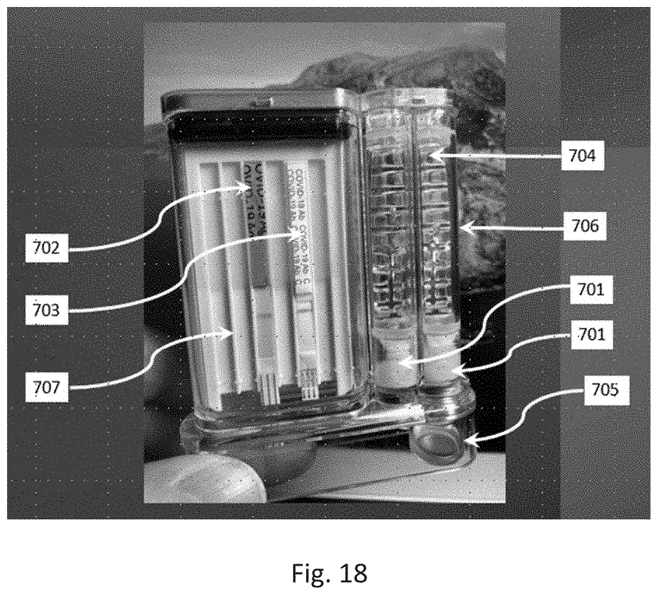

An embodiment of the dual-swab analyte testing device is depicted in FIG. 18. The device includes a dual-swab fluid collector 704 fully inserted into sample receiving member 706. The fluid collector comprises compressed absorbent materials 701 facing downward in the sample receiving member. The device also includes sample retention member 705. As discussed above, the retained portion of the oropharyngeal lavage and be used for further testing, or for confirmation of the test subject's identity through analysis. In this embodiment, a SARS-CoV-2 virus antigen test strip 702 and SARS-CoV-2 antibody test strip 703 are present in the test cartridge 707.

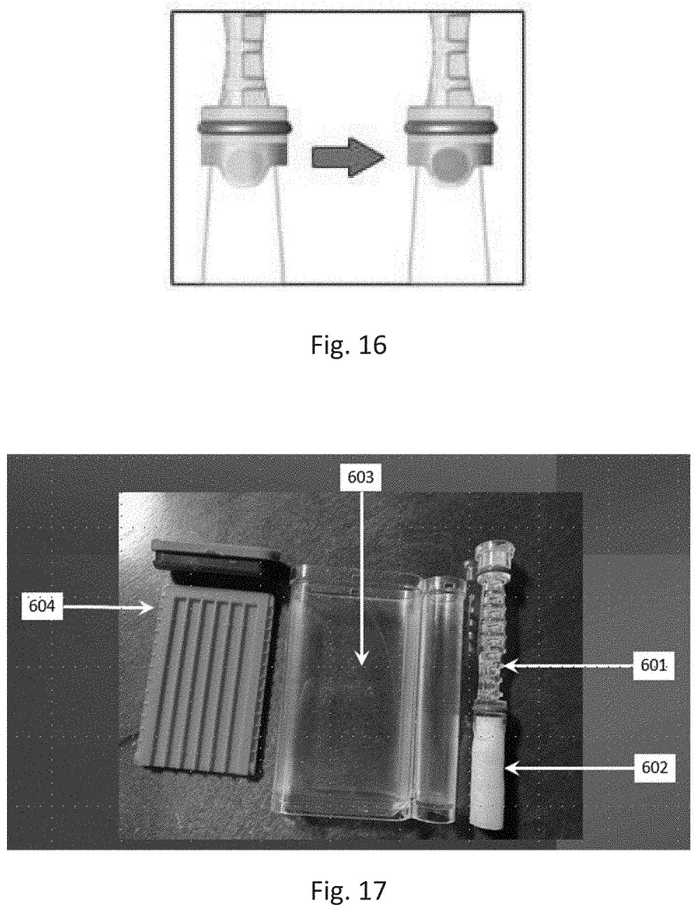

In another beneficial embodiment, the fluid collector can comprise a color indicator that either appears or disappears when a sufficient amount of oropharyngeal lavage is collected by the absorbent material. As discussed above, the absorbent end of the fluid collector is inserted under the tongue of the user and held in place for a fourth period of time, which can be at least one minute. In this embodiment, the absorbent end of the fluid collector is held in place under the tongue of the user until a color indicator appears or disappears. The color indicator is illustrated in FIG. 16 as a circular area. This area should be visible to the user when the absorbent end of the fluid collector is inserted under the tongue, i.e. facing upwards in the user's line of sight.

This disclosure includes at least the following individual embodiments. These and other embodiments of the method described herein are not mutually exclusive, and are independently combinable.

A method of detecting SARS-CoV-2 virus antigen present in oropharyngeal lavage comprises the following steps: abstaining from placing liquid or food in an oral cavity of a user for a first period of time before collecting the oropharyngeal lavage; pouring a non-alcoholic mouth rinse into the oral cavity, and swishing and gargling the non-alcoholic mouth rinse for a second period of time to distribute the non-alcoholic mouth rinse around the inside of the oral cavity and throughout an oropharyngeal space of the user; inserting an absorbent end of a fluid collector comprising an absorbent material into the oral cavity to collect the oropharyngeal lavage from the oral cavity, the oropharyngeal lavage including the non-alcoholic mouth rinse and particles retrieved from the non-alcoholic mouth rinse; removing the absorbent end of the fluid collector from the oral cavity; inserting the fluid collector into the sample receiving member of an analyte testing device for testing a fluid sample, with the absorbent end facing downward; and after a third period of time, reading a first indicator on a first test strip in the device to determine whether SARS-CoV-2 virus antigen is detected in the oropharyngeal lavage.

The analyte testing device used in the method comprises: a sample receiving member having an opening for receiving the oropharyngeal lavage, wherein the sample receiving member includes at least first and second sample collection chambers, and a sample retention member in fluid communication with the first sample collection chamber to retain a portion of the oropharyngeal lavage; and a test cartridge member in fluid communication with only the second sample collection chamber to indicate the presence or absence of SARS-CoV-2 virus antigen in the oropharyngeal lavage; wherein the test cartridge member comprises a test cartridge having a front set of test strip slots and a back set of test strip slots, and at least one test strip to indicate the presence or absence of SARS-CoV-2 virus antigen in the oropharyngeal lavage.

In some embodiments of the method, the method further comprises detecting SARS-CoV-2 virus IgM/IgG antibodies, wherein the test cartridge has a second test strip to indicate the presence or absence of SARS-CoV-2 virus IgM/IgG antibodies.

In some embodiments of the method, the absorbent end of the fluid collector is inserted under the tongue of the user and held in place for a fourth period of time, and wherein the absorbent end of the fluid collector is configured to soak up the entire oropharyngeal lavage of the oral cavity.

In some embodiments of the method, the first period of time is at least 15 minutes; the second period of time is at least 30 seconds; the third period of time is 7 to 10 minutes; and the fourth period of time is at least one minute.

In some embodiments of the method, the fluid collector is inserted into the sample receiving member, absorbent end facing downward, until the fluid collector locks into place and the end of the fluid collector opposite the absorbent end is flush with the top of the sample receiving member.

In some embodiments of the method, the test cartridge member further comprises a test cartridge chamber and a test cartridge fluid reservoir in fluid communication with the sample receiving member, wherein the test cartridge is inserted into the test cartridge chamber and wherein the at least one test strip is in fluid communication with the test cartridge fluid reservoir.

In some embodiments of the method, the test cartridge fluid reservoir is a V-shaped chamber with a flat bottom.

In some embodiments of the method, the V-shaped chamber with a flat bottom forms a volume of less than or equal to four hundred eighty (480) microliters.

In some embodiments of the method, the test cartridge member further comprises a cap to secure the test cartridge.

In some embodiments of the method, the front set of test strip slots comprise at least six test strip slots and wherein the back set of test strip slots comprise at least six test strip slots.

In some embodiments of the method, a plurality of test strips are inserted into the front set of test strip slots and the back set of test strip slots, and wherein the plurality of test strips are in fluid communication with the test cartridge fluid reservoir.

In some embodiments of the method, the analyte testing device is a single-swab analyte testing device and the fluid collector is a single-swab fluid collector having a single prong of absorbent material, wherein the single-swab analyte testing device is configured to receive the single-swab fluid collector.

In some embodiments of the method, the analyte testing device is a multiple-swab analyte testing device and the fluid collector is a multiple-swab fluid collector having multiple prongs of absorbent material, wherein the multiple-swab analyte testing device is configured to receive the multiple-swab fluid collector.

In some embodiments of the method, the analyte testing device is a dual-swab analyte testing device and the fluid collector is a dual-swab fluid collector having a single prong of absorbent material, wherein the dual-swab analyte testing device is configured to receive the dual-swab fluid collector.

In some embodiments of the method, the fluid collector comprises a color indicator that either appears or disappears when a sufficient amount of oropharyngeal lavage is collected by the absorbent material.

In some embodiments of the method, the first and second sample collection chambers are configured to receive a dual swab fluid collector having two prongs of absorbent material.

In some embodiments of the method, the non-alcoholic mouth rinse comprises: a therapeutic antimicrobial mouth rinse; a salt; and a mucolytic agent.

In some embodiments of the method, the non-alcoholic mouth rinse is a stock solution comprising, based on the total volume of the stock solution: 12 to 62% w/v of the therapeutic antimicrobial mouth rinse; 5 to 25% w/v of the salt; and 2 to 8% w/v of the mucolytic agent.

In some embodiments of the method, the non-alcoholic mouth rinse comprises an antimicrobial that is at least one of cetylpyridinium chloride (CPC), chlorhexidine (CHX), or at least one essential oil selected from the group consisting of eucalyptol, menthol, methyl salicylate, and thymol.

In some embodiments of the method, the salt comprises sodium chloride.

In some embodiments of the method, the mucolytic agent is at least one of guaifenesin (MUCINEX™), carbocysteine, erdosteine, mecysteine, bromhexine, hyperosmolar saline, mannitol powder, N-acetyl-L-cysteine (NAC), N-acetylcysteine, fudosteine, dornase alfa (PULMOZYME™), or thymosin β4.

Another method of detecting SARS-CoV-2 virus antigen present in oropharyngeal lavage, the method comprising the following steps: abstaining from placing liquid or food in an oral cavity of a user for a first period of time before collecting the oropharyngeal lavage; pre-soaking the absorbent end of a fluid collector with a non-alcoholic mouth rinse prior to insertion into the oral cavity; inserting the absorbent end of the fluid collector comprising the absorbent material and non-alcoholic mouth rinse into the oral cavity to collect oropharyngeal lavage from the oral cavity, the oropharyngeal lavage including the non-alcoholic mouth rinse and particles retrieved from the oral cavity; removing the absorbent end of the fluid collector from the oral cavity; inserting the fluid collector into the sample receiving member of an analyte testing device for testing a fluid sample, with the absorbent end facing downward; and after a third period of time, reading a first indicator on a first test strip in the device to determine whether SARS-CoV-2 virus antigen is detected in the oropharyngeal lavage.

A fluid collector comprises an absorbent material attached to a piston-like member, wherein the absorbent material is operatively associated with a spring such that compression of the absorbent material results in compression of the spring, and when compression is released, the spring assists return of the absorbent material to the uncompressed state.

In another embodiment, a fluid collector comprises: an upper segment; an absorbent material to absorb an oropharyngeal lavage liquid attached to one end of the upper segment; and a storage member for holding the absorbent material and the oropharyngeal lavage liquid; wherein the oropharyngeal lavage liquid is absorbed into the absorbent material and is inserted into the storage member. A method for collecting analyte from an oral cavity comprises: pre-soaking the absorbent material of the fluid collector with an oropharyngeal lavage liquid prior to insertion into the oral cavity; inserting the absorbent material of the fluid collector comprising the oropharyngeal lavage liquid into the oral cavity; ejecting the oropharyngeal lavage liquid from the absorbent material into the oral cavity; absorbing the ejected oropharyngeal lavage liquid with the absorbent material from the oral cavity; and removing the absorbent material from the oral cavity.

In some embodiments of the above method, the fluid collector comprises an absorbent material attached to a piston-like member operatively associated with a spring such that compression of the absorbent material results in compression of the spring, and when compression is released, the spring assists return of the absorbent material to its uncompressed state; wherein the oropharyngeal lavage liquid is ejected into the oral cavity by compression of the absorbent material and spring; and wherein the oropharyngeal liquid is absorbed from the oral cavity by release of compression of the absorbent material and spring.

In some embodiments of the method, the analyte is a therapeutic drug or metabolite, and the oropharyngeal lavage liquid dissolves the drug or drug metabolite. The therapeutic drug can comprise at least one of cardiac drugs, antibiotics, antiepilectics, bronchodilators, immunosuppressants, anti-cancer drugs, psychiatric drugs, and metabolites thereof.

In some embodiments of this method, the absorbent material is operatively associated with a spring such that compression of the absorbent material results in compression of the spring, and when compression is released, the spring assists return of the absorbent material to the uncompressed state. The spring and absorbent material can be compressed by a piston-like member pushed inward in the direction of the absorbent material. Compression of the piston-like member causes the non-alcoholic mouth rinse to be ejected in the oral cavity, and release of compression on the piston-like member causes the spring and absorbent material to return to their original position, thereby creating suction so that oropharyngeal lavage from the oral cavity is absorbed by the absorbent material.

An embodiment of the fluid collector comprises an absorbent material attached to a piston-like member, wherein the absorbent material is operatively associated with a spring such that compression of the absorbent material results in compression of the spring, and when compression is released, the spring assists return of the absorbent material to the uncompressed state.

Another embodiment of the fluid collector comprises: an upper segment; an absorbent material attached to one end of the upper segment; and a storage member for holding the absorbent material and an oropharyngeal lavage solution; wherein the absorbent material is inserted into the storage member; and wherein the storage member holds the absorbent member and the oropharyngeal lavage solution, wherein the oropharyngeal lavage solution is absorbed by the absorbent material.

Although specific embodiments are disclosed explicitly herein, various modifications and changes can be made without departing from the scope of this disclosure as set forth in the claims below. The combination of embodiments is expressly anticipated, unless the embodiments are specifically mutually exclusive. Any claimed embodiment may include multiple embodiments as disclosed herein. Accordingly, the specification and figures are to be regarded in an illustrative rather than a restrictive sense, and any obvious modifications are intended to be included within the scope of this disclosure. Any benefits, advantages, or solutions to problems that are described herein with regard to specific embodiments are not intended to be construed as a critical, required, or essential feature or element of any or all the claims.

From time-to-time, the disclosure is described herein in terms of exemplary embodiments. The description of these exemplary embodiments is provided to allow the various features to be portrayed in the context of exemplary applications. After reading this disclosure, it will become apparent to one of ordinary skill in the art how the method can be implemented in different and alternative forms. Unless defined otherwise, all technical and scientific terms used herein have the same meaning as is commonly understood by one of ordinary skill in the art to which this disclosure belongs.

The preceding discussion is presented to enable a person skilled in the art to practice the methods disclosed herein. The general principles described herein may be applied to embodiments and applications other than those detailed in the claims without departing from the spirit and scope of the disclosure. The disclosure is not intended to be limited to the embodiments shown, but is to be accorded the widest scope consistent with the principles and features disclosed herein.

In addition, while a particular feature of the method may have been disclosed with respect to only one of several embodiments, such feature may be combined with one or more other features of other embodiments as may be desired. It is therefore contemplated that claims will cover any such modifications that fall within the broad scope of the disclosure.

The various diagrams may depict an exemplary device, composition, or other configuration or the method, which is done to aid in understanding the features and functionality that can be included in the broad scope of the disclosure. The disclosure is not restricted to the illustrated example architectures or configurations, but the desired features can be implemented using a variety of alternative architectures and configurations. Indeed, it will be apparent to one of skill in the art how alternative functional, logical or physical, partitioning and configurations can implement desired features of the method. In addition, a multitude of different component names other than those used herein can be applied to the various components. Additionally, with regard to flow diagrams, operational descriptions and method claims, the order in which the steps are presented herein shall not mandate that various embodiments be implemented to perform the recited steps in the same order unless the context dictates otherwise.

A concentration expressed herein as “% w/v” means weight per volume percent, which is equivalent to g solute per 0.1 L total volume, or g solute per 100 mL total volume.

The terms “about”, “substantially”, “approximately”, “circa”, and variations thereof are intended to include the degree of error associated with measurement of the particular quantity based upon the equipment available at the time of filing the application. For example, “about” a given value can include a range of ±10%, ±5%, or ±1% of the given value.

References to numerical ranges with lower and upper endpoints herein include all numbers subsumed within the range (including fractions), whether explicitly recited or not, as well as the endpoints of the range. Thus, “1 to 5” includes 1, 2, 3, 4, and 5 when referring to, for example, a number of elements, and can also include 1.5, 2, 2.75, 3.8, or any other decimal amount when referring to, for example, quantitative measurements.

“At least one of” in connection with a list means that the list is inclusive of each element individually, as well as combinations of two or more elements of the list, as well as combinations of at least one element of the list with like elements not named.

Terms and phrases used in this disclosure, and variations thereof, unless otherwise expressly stated, should be construed as open ended as opposed to limiting. As examples of the foregoing: the term “including” should be read as meaning “including, without limitation” or the like; the term “example” is used to provide exemplary instances of the relevant item, not an exhaustive or limiting list thereof; the terms “a” or “an” should be read as meaning “at least one”, “one or more” or the like. The presence of broadening words and phrases such as “one or more”, “at least”, “but not limited to” or other like phrases shall not be read to mean that the narrower case is intended or required in instances where such broadening phrases may be absent.

A group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the method or analyte testing device may be described or claimed in the singular, the plural is contemplated to be within the scope thereof unless limitation to the singular is explicitly stated.

Unless stated otherwise, terms such as “first” and “second” are used to arbitrarily distinguish between the elements such terms describe. Thus, these terms are not necessarily intended to indicate temporal or other prioritization of such elements.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

All publications and patents mentioned in the above specification are herein incorporated by reference. Various modifications and variations of the method and analyte testing device disclosed herein will be apparent to those skilled in the art without departing from the scope and spirit of this disclosure. Although the method has been described in connection with specific representative embodiments, it should be understood that the method as claimed should not be unduly limited to such specific embodiments. Indeed, various modifications of the described modes for carrying out the method which are obvious to those skilled in the field or any related fields are intended to be within the scope of the following claims.

One skilled in the art will recognize that different embodiments may be formed in a similar manner having different characteristics depending upon need, performance, or some other criteria. It will thus be appreciated by those skilled in the art that changes could be made to the embodiments described herein without departing from the broad inventive concept thereof. It is understood, therefore, that the method disclosed herein is not limited to the particular embodiments disclosed, but it is intended to cover modifications within the spirit and scope of the present disclosure as defined by the appended claims.

Although this disclosure has been described in conjunction with specific embodiments, many alternatives, modifications and variations will be apparent to those skilled in the art. Accordingly, the embodiments of the method as set forth herein are intended to be illustrative, not limiting. Various changes may be made without departing from the true spirit and full scope of the disclosure as set forth herein.

What is claimed is:

1. A method of detecting SARS-CoV-2 virus antigen present in oropharyngeal lavage, the method comprising the following steps:
abstaining from placing liquid or food in an oral cavity of a user for a first period of time before collecting the oropharyngeal lavage;
pouring a non-alcoholic mouth rinse into the oral cavity, and swishing and gargling the non-alcoholic mouth rinse for a second period of time to distribute the non-alcoholic mouth rinse around the inside of the oral cavity and throughout an oropharyngeal space of the user;
inserting an absorbent end of a fluid collector comprising an absorbent material into the oral cavity to collect the oropharyngeal lavage from the oral cavity, the oropharyngeal lavage including the non-alcoholic mouth rinse and particles retrieved from the non-alcoholic mouth rinse;
removing the absorbent end of the fluid collector from the oral cavity;
inserting the fluid collector into the sample receiving member of an analyte testing device for testing a fluid sample, with the absorbent end facing downward; and
after a third period of time, reading a first indicator on a first test strip in the device to determine whether SARS-CoV-2 virus antigen is detected in the oropharyngeal lavage.

2. A method of detecting SARS-CoV-2 virus antigen present in oropharyngeal lavage, the method comprising the following steps:
abstaining from placing liquid or food in an oral cavity of a user for a first period of time before collecting the oropharyngeal lavage;
pre-soaking the absorbent end of a fluid collector with a non-alcoholic mouth rinse prior to insertion into the oral cavity;
inserting the absorbent end of the fluid collector comprising the absorbent material and non-alcoholic mouth rinse into the oral cavity to collect oropharyngeal lavage from the oral cavity, the oropharyngeal lavage including the non-alcoholic mouth rinse and particles retrieved from the oral cavity;
removing the absorbent end of the fluid collector from the oral cavity;
inserting the fluid collector into the sample receiving member of an analyte testing device for testing a fluid sample, with the absorbent end facing downward; and
after a third period of time, reading a first indicator on a first test strip in the device to determine whether SARS-CoV-2 virus antigen is detected in the oropharyngeal lavage.

3. The method of detecting SARS-CoV-2 virus antigen of claim 2, wherein the absorbent material is operatively associated with a spring such that compression of the absorbent material results in compression of the spring, and when compression is released, the spring assists return of the absorbent material to the uncompressed state.

4. The method of detecting SARS-CoV-2 virus antigen of claim 3, wherein the spring and absorbent material are compressed by a piston-like member pushed inward in the direction of the absorbent material.

5. The method of detecting SARS-CoV-2 virus antigen of claim 4, wherein compression of the piston-like member causes the non-alcoholic mouth rinse to be ejected in the oral cavity, and wherein release of compression on the piston-like member causes the spring and absorbent material to return to their original position, thereby creating suction so that oropharyngeal lavage from the oral cavity is absorbed by the absorbent material.

6. The method of detecting SARS-CoV-2 virus antigen of claim 1, wherein the analyte testing device comprises:
a sample receiving member having an opening for receiving the oropharyngeal lavage, wherein the sample receiving member includes at least first and second sample collection chambers, and a sample retention member in fluid communication with the first sample collection chamber to retain a portion of the oropharyngeal lavage; and
a test cartridge member in fluid communication with only the second sample collection chamber to indicate the presence or absence of SARS-CoV-2 virus antigen in the oropharyngeal lavage;
wherein the test cartridge member comprises a test cartridge having a front set of test strip slots and a back set of test strip slots, and at least one test strip to indicate the presence or absence of SARS-CoV-2 virus antigen in the oropharyngeal lavage.

7. The method of claim 6, further comprising detecting SARS-CoV-2 virus IgM/IgG antibodies, wherein the test cartridge has a second test strip to indicate the presence or absence of SARS-CoV-2 virus IgM/IgG antibodies.

8. The method of claim 1, wherein the absorbent end of the fluid collector is inserted under the tongue of the user and held in place for a fourth period of time, and wherein the absorbent end of the fluid collector is configured to soak up the entire oropharyngeal lavage of the oral cavity.

9. The method of claim 8, wherein:
the first period of time is at least 15 minutes;
the second period of time is at least 30 seconds;
the third period of time is 7 to 10 minutes; and
the fourth period of time is at least one minute.

10. The method of claim 8, wherein the fluid collector is inserted into the sample receiving member, absorbent end facing downward, until the fluid collector locks into place and the end of the fluid collector opposite the absorbent end is flush with the top of the sample receiving member.

11. The method of claim 6, wherein the test cartridge member further comprises a test cartridge chamber and a test cartridge fluid reservoir in fluid communication with the sample receiving member, wherein the test cartridge is inserted into the test cartridge chamber and wherein the at least one test strip is in fluid communication with the test cartridge fluid reservoir.

12. The method of claim 11, wherein the test cartridge fluid reservoir is a V-shaped chamber with a flat bottom.

13. The method of claim 12, wherein the V-shaped chamber with a flat bottom forms a volume of less than or equal to four hundred eighty (480) microliters.

14. The method of claim 1, wherein the test cartridge member further comprises a cap to secure the test cartridge.

15. The method of claim 6, wherein the front set of test strip slots comprise at least six test strip slots and wherein the back set of test strip slots comprise at least six test strip slots.

16. The method of claim 6, wherein a plurality of test strips are inserted into the front set of test strip slots and the back set of test strip slots, and wherein the plurality of test strips are in fluid communication with the test cartridge fluid reservoir.

17. The method of claim 1, wherein the analyte testing device is a single-swab analyte testing device and the fluid collector is a single-swab fluid collector having a single prong of absorbent material, wherein the single-swab analyte testing device is configured to receive the single-swab fluid collector.

18. The method of claim 1, wherein the analyte testing device is a multi-swab analyte testing device and the fluid collector is a multiple-swab fluid collector having multiple prongs of absorbent material, wherein the multiple-swab analyte testing device is configured to receive the multiple-swab fluid collector.

19. The method of claim 18, wherein the analyte testing device is a dual-swab analyte testing device and fluid collector is a dual-swab fluid collector having two prongs of absorbent material, wherein the dual-swab analyte testing device is configured to receive the dual-swab fluid collector.

20. The method of claim 1, wherein the fluid collector comprises a color indicator that either appears or disappears when a sufficient amount of oropharyngeal lavage is collected by the absorbent material.

21. The method of claim 6, wherein the first and second sample collection chambers are configured to receive a dual swab fluid collector having two prongs of absorbent material.

22. The method of claim 1, wherein the non-alcoholic mouth rinse comprises:
a therapeutic antimicrobial mouth rinse;
a salt; and
a mucolytic agent.

23. The method of claim 18, wherein the non-alcoholic mouth rinse is a stock solution comprising, based on the total volume of the stock solution:
12 to 62% w/v of the therapeutic antimicrobial mouth rinse;
5 to 25% w/v of the salt; and
2 to 8% w/v of the mucolytic agent.

24. The method of claim 22, wherein the non-alcoholic mouth rinse comprises an antimicrobial that is at least one of cetylpyridinium chloride (CPC), chlorhexidine (CHX), or at least one essential oil selected from the group consisting of eucalyptol, menthol, methyl salicylate, and thymol.

25. The method of claim 22, wherein the salt comprises sodium chloride.

26. The method of claim 22, wherein the mucolytic agent is at least one of guaifenesin (MUCINEX™), carbocysteine, erdosteine, mecysteine, bromhexine, hyperosmolar saline, mannitol powder, N-acetyl-L-cysteine (NAC), N-acetylcysteine, fudosteine, dornase alfa (PULMOZYME™), or thymosin β4.

27. A fluid collector comprising an absorbent material attached to a piston-like member, wherein the absorbent material is operatively associated with a spring such that compression of the absorbent material results in compression of the spring, and when compression is released, the spring assists return of the absorbent material to the uncompressed state.

28. A fluid collector comprising:
an upper segment;
an absorbent material to absorb an oropharyngeal lavage liquid attached to one end of the upper segment; and
a storage member for holding the absorbent material and the oropharyngeal lavage liquid;
wherein the oropharyngeal lavage liquid is absorbed into the absorbent material and is inserted into the storage member.

29. A method for collecting analyte from an oral cavity comprising:
pre-soaking the absorbent material of the fluid collector of claim 28 with an oropharyngeal lavage liquid prior to insertion into the oral cavity;
inserting the absorbent material of the fluid collector comprising the oropharyngeal lavage liquid into the oral cavity;
ejecting the oropharyngeal lavage liquid from the absorbent material into the oral cavity;
absorbing the ejected oropharyngeal lavage liquid with the absorbent material from the oral cavity; and
removing the absorbent material from the oral cavity.

30. The method of claim 29, wherein the fluid collector comprises an absorbent material attached to a piston-like member operatively associated with a spring such that compression of the absorbent material results in compression of the spring, and when compression is released, the spring assists return of the absorbent material to its uncompressed state;
wherein the oropharyngeal lavage liquid is ejected into the oral cavity by compression of the absorbent material and spring; and wherein the oropharyngeal liquid is absorbed from the oral cavity by release of compression of the absorbent material and spring.

31. The method of claim 29, wherein the analyte is a therapeutic drug or metabolite, and the oropharyngeal lavage liquid dissolves the drug or drug metabolite.

32. The method of claim 31, wherein the therapeutic drug comprises at least one of cardiac drugs, antibiotics, antiepilectics, bronchodilators, immunosuppressants, anti-cancer drugs, psychiatric drugs, and metabolites thereof.
